(19) United States
(12) Patent Application Publication
(10) Pub. No.: US 2009/0264421 A1
(43) Pub. Date: Oct. 22, 2009

(54) Methods and Compositions for Treating Cancer

(76) Inventors: Keith C. Bible, Rochester, MN (US); Crescent R. Isham, Rochester, MN (US); Ruifang Xu, Naperville, IL (US); Jennifer D. Tibodeau, Chanhassen, MN (US)

Correspondence Address: FISH & RICHARDSON P.C., PO BOX 1022, MINNEAPOLIS, MN, 55440-1022, US

(21) Appl. No.: 12/406,444

(22) Filed: Mar. 18, 2009

(30) Foreign Application Priority Data
Oct. 5, 2007 (US) PCT/US2007/080588

(51) Int. Cl. A61K 31/548; C07D 513/08; A61P 35/00

(52) U.S. Cl. 514/222.8; 544/5

(57) ABSTRACT

Compositions and methods for treating cancer, including hematologic cancers such as multiple myeloma, are disclosed. In some embodiments, chaetocin, chaetomin, or gliotoxin can be used to treat or ameliorate one or more symptoms or disorders associated with multiple myeloma.

### CROSS-REFERENCE TO RELATED APPLICATIONS

This document is a continuation-in-part application of and claims priority to PCT/US2007/080588, filed on Oct. 5, 2007, which claims priority to U.S. Application Ser. No. 60/849,576, filed on Oct. 5, 2006, by Bible et al., and entitled “Methods and Compositions for Treating Cancer.” The contents of each document are herein incorporated by reference in their entirety.

### STATEMENT AS TO FEDERALLY FUNDED RESEARCH

The inventions were made with government support under grants from the NIH and the NCI (R01 CA97129 and R01 CA98118). The Government has certain rights in the inventions.

### TECHNICAL FIELD

This disclosure relates to compositions and methods for treating hematologic cancers, including myelomas, leukemias, and lymphomas, and solid cancerous tumors using thiodioxopiperazines, e.g., bridged disulfide thiodioxopiperazines, also known as epipolythiodioxopiperazines, and structurally related compounds.

### BACKGROUND

Chaetocin, a small molecule natural product produced by Chaetomium species fungi and originally isolated in 1970 (Hauser D, Weber H P, and Sigg H P, Isolation and Configuration of Chaetocin, Helv Chim Acta (1970) 53(5):1061-73) is a representative of a class of fungal secondary metabolites known as thiodioxopiperazines. Other thiodioxopiperazines have been previously reported to have a wide range of biological activities, including antimicrobial and antifungal effects. Thiodioxopiperazines may be produced by fungi to gain a competitive advantage over adjacent fungal and other saprophytic organisms through toxic and antiproliferative effects on adjacent organisms.

Thioredoxin reductase-1 (TrxR1) is a 55 kD per subunit homodimeric protein belonging to a family of glutathione reductase-like flavoenzymes. TrxR1 catalyzes the NADPH-dependent reduction of thioredoxin and other substrate disulfide bonds via its selenocysteine/FAD active site. Mammalian TrxR1 consequently participates in diverse metabolic reactions involving oxidation-reduction cycles and is widely believed to be central to intracellular ROS mitigation.

The TrxR1/Trx pathway may provide plausible molecular targets for cancer therapies for several reasons. First, TrxR1 and/or Trx are known to be upregulated in a variety of human cancers, including lung, colorectal, cervical, hepatic, and pancreatic, and Trx overexpression has been linked to aggressive tumor growth and poorer prognosis. Second, TrxR1 enhances tumor proliferation via its regulatory effects on the G₁ checkpoint during cell cycle progression. Third, TrxR1 invokes a pro-survival signaling cascade. Further, cells overexpressing TrxR1 are more resistant to some anticancer agents. Moreover, upregulated TrxR1/Trx pathway activity may in part account for how cancer cells have adapted to their generally higher basal levels of cellular oxidative stress. Therefore, despite providing a potential survival advantage to cancer cells, upregulated TrxR1/Trx pathway activity may also be required for cancer survival in light of increased ROS stress inherent in some cancer cells. In this fashion, the TrxR1/Trx pathway may contain therapeutically useful antineoplastic molecular targets.

Small molecules such as lipid hydroperoxides, selenite and dehydroascorbate, as well as proteins such as protein disulfide isomerase or glutathione peroxidase along with Trx, are all known substrates of TrxR1, demonstrating its low substrate specificity. There are several known inhibitors of TrxR1 including auranofin, cisplatin, lipoic acid, motexafin gadolinium, myricetin, quercetin, and 1-methyl-1-propyl-2-imidazolyl disulfide (IV-2). Of these, motexafin gadolinium and IV-2 have anticancer effects putatively attributed to TrxR1 inhibition and are undergoing development as candidate cancer therapeutics.

Multiple myeloma is an incurable cancer characterized by the clonal proliferation of B-cell lineage plasma cells, resulting in the production of monoclonal proteins in serum and/or urine and destructive boney lesions, and the deaths of about 12,000 individuals in the U.S. annually. Although increasing numbers of therapeutics are becoming available to treat this disease—with the potential for significant symptom palliation, induction of disease responses, and prolongation of disease-free survival—available therapeutic approaches including peripheral blood stem cell transplantation and newer therapeutics have yet to be conclusively demonstrated to appreciably impact patient overall survival in randomized trials (Barlogie et al., 2005; Fermand et al, 2005; Blade et al., 2005). As a consequence, there is still need for improved anti-myeloma therapies.

### SUMMARY

This disclosure is based, in part, on the discovery that chaetocin, a thiodioxopiperazine natural product, is a potential anti-cancer therapeutic. Chaetocin has potent in vitro anti-myeloma activity in IL-6-dependent and IL-6-independent myeloma cell lines. Chaetocin potently killed freshly collected sorted patient CD138+ myeloma cells, but spared matched normal CD138− patient bone marrow leukocytes, and displayed superior ex vivo anti-myeloma activity and selectivity in comparison to doxorubicin and dexamethasone. In vivo experiments using chaetocin confirmed the antiproliferative activity in myeloma. Furthermore, the effects of chaetocin were seen in samples obtained from patients afflicted with different types of myeloma, including smoldering myeloma and heavily pretreated myeloma patients who had previously undergone peripheral blood stem cell transplantation.

Mechanistically, chaetocin is rapidly and dramatically accumulated in cancer cells via a transport system inhibited by glutathione and requiring intact/unreduced disulfides for uptake. Once inside the cell, its anti-cancer activity appears mediated primarily through the imposition of oxidative stress and apoptosis induction. The ability of chaetocin to selectively kill myeloma cells appears to be mediated based upon a generally increased susceptibility of myeloma cells to oxidative stressors. This suggests that not only chaetocin, but also other agents that similarly induce cellular oxidative stress, may hold promise for further development as potential anti-myeloma therapeutics.

More specifically, chaetocin is a competitive and selective substrate for, and inhibitor of, the oxidative stress mitigation enzyme thioredoxin reductase-1 (TrxR1), with a lower Kₘ than the TrxR1 native substrate thioredoxin (Trx; chaetocin Kₘ=4.6±0.6 μM, Trx Kₘ=104.7±26 μM). Consequently, chaetocin thereby competitively inhibits TrxR1 reduction of the critical TrxR1 downstream ROS remediation substrate Trx at achievable intracellular concentrations. Indeed, transient overexpression of the downstream TrxR1 effector Trx rescues HeLa cancer cells from chaetocin-induced, but not doxorubicin-induced, cell death. As the TrxR1/Trx pathway is of central importance in limiting reactive oxygen species (ROS) accumulation in cancer cells, and as chaetocin exerts its selective anti-myeloma effects via ROS imposition, the inhibition of TrxR1 by chaetocin appears to link enzyme targeting of TrxR1 by chaetocin to chaetocin's selective anti-myeloma effects. The present data thus demonstrate the link between chaetocin's imposition of cellular oxidative stress in myeloma cells to chaetocin's effects on a specific molecular target, TrxR1

Based on this data, structurally related compounds (e.g., those containing intact disulfides, those containing a bridged disulfide thiodioxopiperizine ring, and/or those capable of inducing oxidative stress) are proposed to have similar anti-cancer activities as chaetocin. Examples of structurally related compounds that include a bridged disulfide thiodioxopiperazine ring include chaetomin and gliotoxin. The inventors have found that chaetomin and gliotoxin also inhibit the reduction of thioredoxin by thioredoxin reductase. Other examples of compounds that have a bridged disulfide thiodioxopiperazine ring include melinacidin IV, sirodesmin, hyalodendrin, sporidesmin A, leptosin, emestrin, dithiosilvatin, epicorazine, emethallicin, verticillin A and B, 19 methyl-19′deoxy-6,6′dihydroxychaetocin, aranotin, apoaranotin, and scabrosin. All of these compounds thus may be useful in the described methods for treating cancer, e.g., myeloma.

Accordingly, compounds, compositions, and methods for treating a cancer are provided herein. In some embodiments, the cancer is multiple myeloma. In other embodiments, the cancer is a solid tumor.

In one embodiment, provided is a composition comprising a compound according to Formula I:

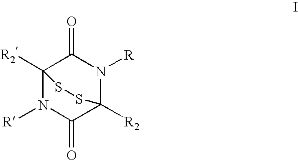

or a pharmaceutically acceptable salt or derivative thereof, where R, R′, R₂, and R₂′, independently, can be selected from the group consisting of hydrogen, alkyl, cycloalkyl, alkoxy, hydroxy, aryl, heteroaryl, heteroalkyl, heterocyclyl, halo, pseudohalo, carboxy, haloalkyl, hydroxyalkyl, alkaryl, aralkyl, aminocarbonyl, aminoalkyl, alkylcarbonyl, arylcarbonyl, alkoxy, alkylthio, aryloxy, and arylthio; or

wherein R and R₂, and/or R′ and R₂′, independently, together to which the atoms to which they are attached, form a fused heterocyclic ring system which is optionally substituted with one or more alkyl, cycloalkyl, alkoxy, aryl, hydroxy, heteroaryl, heteroalkyl, heterocyclyl, halo, pseudohalo, carboxy, haloalkyl, hydroxyalkyl, alkaryl, aralkyl, aminocarbonyl, aminoalkyl, alkylcarbonyl, arylcarbonyl, alkoxy, alkylthio, aryloxy, or arylthio groups, or another optionally substituted aryl, heterocyclyl, or cycloalkyl fused ring system,

provided that R and R′ are not substituted with the same group when R₂ and R₂′ are both H, and provided that if R and R₂ form a fused ring heterocyclic ring system, then R′ and R₂′ do not also form a fused ring heterocyclic system, and if R and R₂ form a fused ring heterocyclic ring system and R′ is methyl, then R₂′ is not hydroxyalkyl or alkyl.

In certain embodiments, R and R′ are selected independently from hydrogen, halo, pseudohalo, hydroxyl, alkyl having from 1 to 8 carbon atoms, aralkyl, where the alkyl group has from 1 to 8 carbon atoms, alkaryl, where the alkyl group has from 1 to 8 carbon atoms, hydroxyalkyl, where the alkyl group has from 1 to 8 carbon atoms, alkylcarbonyl, where the alkyl group has from 1 to 8 carbon atoms, and arylcarbonyl.

In certain embodiments, R and R′ are independently selected from hydrogen, methyl, ethyl, hydroxymethyl, hydroxyethyl, and hydroxypropyl.

In certain embodiments, R₂ and R₂′ are independently selected from hydrogen, halo, pseudohalo, alkyl having from 1 to 8 carbon atoms, and hydroxyalkyl, where the 5 alkyl group has from 1 to 8 carbon atoms.

In certain embodiments, R and R′ are H.

Certain embodiments provide a composition of matter comprising a compound having the structure of chaetocin, chaetomin, or gliotoxin, or a pharmaceutically acceptable salt or derivative thereof, and an anticancer drug.

Certain embodiments provide a method of treating or ameliorating one or more symptoms associated with cancer in a mammal, comprising administering a composition according to claim 1 or claim 6, or a composition comprising chaetocin, chaetomin, or gliotoxin, or a pharmaceutically acceptable salt or derivative thereof, to the mammal.

In certain embodiments, the cancer overexpresses TrxR1. In certain embodiments, the cancer is a hematologic cancer or disorder, multiple myeloma, or B-lymphocyte lineage malignancy or disorder.

In certain embodiments, the cancer is a leukemia or a lymphoma.

Certain embodiments provide a method for inducing oxidative stress in a cancerous cell comprising contacting the cancerous cell with a composition according to claim 1 or claim 6, or a composition comprising chaetocin, chaetomin, or gliotoxin, or a pharmaceutically acceptable salt or derivative thereof. In certain embodiments, the cancer overexpresses TrxR1.

In certain embodiments, the cancerous cell is a multiple myeloma cell.

In certain embodiments, the oxidative stress is measured via a fluorescent assay. In certain embodiments, the fluorescent assay employs the fluorescent probe 5,6-carboxy-2′,7′-difluoro-dihydro-fluorescein diacetate.

Certain embodiments provide a method for inducing apoptosis in a cancerous cell comprising contacting the cancerous cell with a composition according to claim 1 or claim 6, or a composition comprising chaetocin, chaetomin, or gliotoxin, or a pharmaceutically acceptable salt or derivative thereof.

Certain embodiments provide a kit comprising any one of the aforementioned compositions. In certain embodiments, the composition is in the form of an injectible composition.

Certain embodiments provide a composition according to any one of the aforementioned compositions, or comprising chaetocin, chaetomin, or gliotoxin, or a pharmaceutically acceptable salt or derivative thereof, for use in the treatment or amelioration of cancer in a mammal. In certain embodiments, the cancer is multiple myeloma.

Certain embodiments provide a article of manufacture comprising a composition according to any one of the aforementioned compositions within a pill, a tablet, a capsule, or a syringe.

Certain embodiments relate to any of the aforementioned methods, where the cancer is a solid tumorous cancer. In certain embodiments, the solid tumorous cancer is selected from the group consisting of lung, breast, prostate, hepatoma, thyroid, colon, cervical, pancreatic, and sarcoma solid tumors.

Certain embodiments relate to any one of the aforementioned methods, where the cancer is a B-lymphocyte lineage malignancy or disorder is selected from MGUS, amyloidosis, heavy chain diseases, cryoglobulinemia, and Waldenstrom's macroglobulinemia.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Chaetocin bears chemical structural similarity to the acetylated histone lysine moiety mimicked by many histone deacetylase inhibitors (HDACIs) and has anti-myeloma activity in vitro—yet does not alter levels of acetylated histone H3 in myeloma cells. A. The chemical structure of chaetocin, indicating similarities to the acetylated histone lysine moiety. B. and C. Effects of various chaetocin concentrations (24 hour drug exposures) on survival of KAS-6 IL-6-dependent myeloma cells (B) or OCI-MY5 IL-6-independent myeloma cells (C) in vitro. Cell viability was assessed using a trypan blue exclusion assay with manual counting of cells using a hemocytometer. Displayed results are representative of three independent experiments. D. Effects of Chaetocin on the cellular levels of acetylated histone H3 in A549 human nonsmall cell lung cancer cells. E. Effects of chaetocin or the known HDACIs aphidicolin, LAQ-824 or trichostatin A on the cellular levels of acetylated histone H3 in U266 human myeloma cells. In D. and E. levels of acetylated histone H3 and actin were assessed using immunoblotting of whole cell lysates (50 μg total cellular proteins loaded per lane, 24 hour drug exposures).

FIG. 2. Chaetocin kills myeloma cells in vitro via induction of morphological apoptosis accompanied by loss of mitochondrial membrane potential, PARP cleavage and DNA ladder formation. A. Transmission electron photomicrographs of OCI-MY5 myeloma cells treated with DMSO or 100 nM chaetocin for 24 hours. B. Effects of 24 hour exposure of OCI-MY5 myeloma cells to varying chaetocin concentrations on mitochondrial membrane depolarization (shaded bars) and apoptosis (filled circles and lines). Mitochondrial membrane potential was assessed via FACS, while apoptosis was assessed via fluorescence microscopy utilizing Hoechst 33256 staining as described below. Results shown are representative of four independent experiments. C. Induction of PARP cleavage in KAS 6/1 and OCI-MY5 myeloma cells by 100 nM chaetocin (time course). Upper bands represent the results of PARP immunoblotting, while the lower band indicates actin control immunoblotting. Results shown are representative of four independent experiments. D. Electrophoresis of DNA extracted from diluent- or chaetocin-treated OCI-MY5 myeloma cells for 24 hours. Bands corresponding to uncleaved DNA, and cleaved DNA “ladders” are indicated at the right. Results shown are representative of three independent experiments.

FIG. 3. Chaetocin kills freshly collected sorted patient CD138+ myeloma cells with superior efficacy and selectivity than the commonly utilized anti-myeloma agents dexamethasone and doxorubicin—while sparing matched normal CD138− bone marrow leukocytes, normal B-cells and neoplastic B-CLL (chronic lymphocytic leukemia) cells. Results from three representative myeloma patients (of 20 similarly assessed) are displayed, designated A (M-1), B (M-2) and C (M-3). Patient A (M-1) had therapy-refractory myeloma, having failed prior treatment with thalidomide, dexamethasone and bortezomib (labeling index=0.6%); Patient B (M-2) had previously untreated smoldering myeloma (labeling index=1%); Patient C (M-3) had myeloma currently undergoing treatment with dexamethasone with stable disease (labeling index=0.2%). D. Treatment of normal B-cells or matched negatively selected neutrophils from three normal patients with chaetocin indicates that, unlike myeloma cells, normal B-cells are not selectively killed by chaetocin. E. Treatment of B-CLL cells or matched negatively selected patient neutrophils from two B-CLL patients with chaetocin also indicates that, unlike myeloma cells, B-CLL cells are not selectively killed by chaetocin. Survival indicated in A-E was assessed using a trypan blue exclusion assay, counting viable cells using a hemocytometer after 24 hour exposure to the indicated drugs and drug concentrations.

FIG. 4. Patient myeloma cells treated in mixed culture are selectively killed by chaetocin in comparison to other bone marrow leukocytes, and chaetocin has in vivo anti-myeloma activity. A-C. Unsorted bone marrow leukocytes obtained from 4 patients with multiple myeloma were treated with 100 nM Chaetocin or diluent for 24 hours and then subjected to FACS analyses to examine induced cell death in various leukocyte subpopulations. Whereas myeloma cells were readily killed by chaetocin (A, representative data shown), combined granulocytes and monocytes (B, representative data shown) were relatively spared. Results from 4 unsorted patient marrow leukocyte samples are indicated in C, indicating selective killing of myeloma cells by chaetocin in mixed culture in all 4 examined patient samples. In A-C, surviving cells were defined as those with low annexin and 7-AAD staining. D. Chaetocin has in vivo anti-myeloma activity in the RRMI 8226 SCID flank xenograft mouse model. Arrows indicate times of intraperitoneal chaetocin administration, while * indicates statistically significance differences from corresponding vehicle control values (p<0.05).

FIG. 5. Chaetocin is rapidly and dramatically accumulated unaltered in cancer cells by means that require intact/unreduced chaetocin disulfide bonds. A. HPLC tracing indicating results of assessment of levels of intracellular chaetocin in chaetocin-treated A549 cells as described below. Note that only a single new HPLC resulted from the treatment of A549 cells with chaetocin, and that this peak corresponds to that of unaltered/unreduced chaetocin. B. Assessment of intracellular (A549 cells) and extracellular (media) chaetocin concentrations as functions of time in response to addition of 10 μM chaetocin to tissue culture media at time 0. C. Time course of intracellular accumulation of chaetocin in A549 cells. For B and C, chaetocin concentrations were determined as described in the text using HPLC. D. Effects of chaetocin, dithiothreotol-reduced chaetocin or S-methyl chaetocin on colony formation in A549 cells, indicating loss of cytototoxicity upon modification of the chaetocin disulfide bond. Cells were exposed to all agents for 24 hours, with colonies assessed 6-7 days subsequently as described below.

FIG. 6. Glutathione dramatically attenuates chaetocin-induced reductions in A549 cell colony formation, partially mediated through attenuation of intracellular accumulation of chaetocin. A. Glutathione or NAC, but not inhibitors of DNA (aphidicolin), RNA (DRB) or protein (cycloheximide) synthesis, attenuate chaetocin-induced reductions in colony formation in A549 cells. Results shown are representative of three independent experiments for each treatment. B. The ability of glutathione to attenuate chaetocin-induced inhibition of colony formation is highly time-dependent, and is maximal when glutathione is added before initiation of chaetocin exposure. Results shown are representative of two independent experiments. C. and D. The effects of glutathione pretreatment on intracellular (C) and extracellular (media, D) chaetocin concentration in response to treatment of A549 cells with 10 μM chaetocin for 5 min. Intracellular and extracellular chaetocin concentrations were assessed using HPLC as described in the text, with 100 μM glutathione added 5 minutes before chaetocin addition. E. “Double reciprocal” plot indicating the effects of 5 minute pretreatment with 100 mM glutathione or diluent on intracellular chaetocin concentrations resulting from exposure of A549 cells to varying concentration of chaetocin for 5 minutes. Intracellular chaetocin concentrations were assessed using HPLC as described in the text. Results shown are representative of three independent experiments.

FIG. 7. Chaetocin induces oxidative stress in A549 cells without appreciable depletion of intracellular reduced glutathione levels, while the selective cytotoxicity of chaetocin in freshly collected myeloma cells appears to be attributable to their increased sensitivity to oxidative stressors. Treatment of A549 cells with 200 μM hydrogen peroxide (A, positive control) or 400 nM chaetocin (B) for 24 hours resulted in increased intracellular oxidative species as assessed by FACS analyses. Chaetocin-induced increased oxidative species are attenuated by co-treatment with 10 mM NAC (C). D. Summary of results from FACS indicating changes of oxidative species induced in response to various treatments. Results shown are representative of three independent FACS experiments as described in the text; *p<0.05, **p<0.01. E. Effects of chaetocin or hydrogen peroxide (in comparison to diluent) on ROS (superoxide) levels in U266 myeloma cells as assessed using FACS analyses and hydroethidium as an ROS-sensitive FACS probe. Error bars indicate one sample standard deviation, results replicated in triplicate; ***p<0.001. F. Chaetocin did not appreciably alter intracellular concentrations of reduced glutathione, yet pretreatment with 1 mM glutathione led to increased levels of intracellular reduced glutathione. A549 cells were exposed to the indicated concentration of chaetocin for 24 hours, with addition of glutathione or diluent 30 minutes prior to chaetocin addition. Glutathione levels were assessed by spectrophotometric assay as described below. G Relative intracellular chaetocin levels in patient normal CD138− bone marrow cells compared to those attained in identically treated patient CD138+ myeloma cells. Cells were treated with 10 μM chaetocin for 20 minutes prior to assay, and calculated intracellular chaetocin levels were measured by HPLC with adjusted for differences in average cell volume (calculated from measured cell radii ascertained via light microscopy). Shaded area represents 1 standard deviation confidence interval, with central bar reflecting the mean relative intracellular chaetocin level. H. CD138+ patient myeloma cells are more sensitive to the cytotoxic effects of hydrogen peroxide than matched normal patient CD138− bone marrow leukocytes. Cells from 4 myeloma patients were exposed to 200 μM hydrogen peroxide for 24 hours prior to assay, with trypan blue exclusion used to assess surviving cells.

FIG. 8 shows the structures of chaeotocin, chaetomin, and gliotoxin.

FIG. 9 shows a representative synthetic scheme for preparing certain compounds according to Formula I.

FIG. 10 shows the effects of chaetocin on colony formation in solid tumor cancer cell lines. A. Effects of chaetocin on colony formation in a variety of solid tumor cancer cell lines (24 h chaetocin exposures). B. Time-dependence of chaetocin-induced reductions in colony formation in A549 cells. C. Effects of chaetocin on colony formation in a variety of thyroid cancer cell lines (24 h chaetocin exposures).

FIG. 11 demonstrates the effects of chaetocin on normal cells. A and B. Effects of chaetocin on survival of MGUS plasma cells and matched patient normal leucocytes from two patients (assessed by trypan blue exclusion assay, 24 h chaetocin exposures). C and D. Effects of chaetocin on survival of normal plasma cells and matched patient normal leucocytes from two patients (assessed by trypan blue exclusion assay, 24 h chaetocin exposures).

FIG. 12 demonstrates the effects of chaetocin in a variety of thyroid cancer cell lines and in normal thyroid gland thyrocytes. Presented data represent the results of colony forming assays, with 24 hour exposures to chaetocin at the indicated concentrations. Note that normal thyrocytes are distinctly less sensitive to the cytotoxic effects of chaetocin in comparison to all tested thyroid cancer cell lines.

FIG. 13 shows results from assessment of the effects of chaetocin in dexamethasone- or doxorubicin-resistant myeloma cell lines. In particular, dexamethasone-resistant MM1R cells are not cross-resistant to chaetocin (A and B), while doxorubicin-resistant RPMI 8226 D40 cells are only modestly cross-resistant to chaetocin (C and D). Cell numbers and viability were assessed by trypan exclusion.

FIG. 14 demonstrates that chaetocin inhibits thioredoxin reductase activity—but not the activities of glutathione reductase or thioredoxin. A. Chemical structure of chaetocin. B. Thioredoxin reductase activity (assessed by following DTNB reduction) is inhibited by chaetocin in a dose-dependent fashion. Inset. Time versus absorbance data showing inhibition of DTNB reduction by chaetocin at various concentrations and times. Glutathione reductase activity (C, assessed by following DTNB reduction) and thioredoxin activity (D, assessed using the insulin precipitation method) are unaffected by chaetocin at concentrations that readily inhibit thioredoxin reductase. Data shown represent single experiments (triplicate data points; error bars, one standard deviation) representative of a minimum of three separate experiments.

FIG. 15 demonstrates that chaetocin and other intact thiodioxopiperazines inhibit the ability of thioredoxin reductase to reduce its native substrate thioredoxin. A. Chaetocin inhibits the reduction of thioredoxin by thioredoxin reductase in a dose-dependent fashion. B. Chaetocin, chaetomin and gliotoxin each inhibit the reduction of thioredoxin by thioredoxin reductase; however, the related compounds S-methyl chaetocin and verruculogen lacking bridged disulfide bonds do not. C. In a representative initial rate experiment, the IC₅₀ of chaetocin inhibiting redox cycling of thioredoxin by thioredoxin reductase is 4 μM. Data shown are representative of a minimum of three separate experiments, insets show primary gel data.

FIG. 16 shows that chaetocin is a substrate for thioredoxin reductase. A. Oxidation of NADPH by thioredoxin reductase in response to various chaetocin concentrations in the absence of other thioredoxin reductase substrates, indicating that chaetocin itself has thioredoxin reductase substrate functionality. Data shown are representative of a minimum of three separate experiments. B. Mass spectrometry results demonstrating that chaetocin disulfide bonds are reduced in the presence of thioredoxin reductase and NADPH, but not NADPH alone. Note the addition of two (m/z 699) and four (m/z 701) Daltons to chaetocin (m/z 697), indicative of the addition of two or four protons respectively, and reflective of reduction of one or both chaetocin disulfide bonds.

FIG. 17 demonstrates that transient overexpression of the downstream thioredoxin substrate and effector thioredoxin attenuates chaetocin-induced but not doxorubicin-induced cytotoxicity—thus demonstrating the potential impact of chaetocin on reduction states of critical downstream targets of thioredoxin reductase. A. Transient overexpression of thioredoxin attenuates chaetocin—but not doxorubicin-induced cytotoxicity in HeLa cells. Transfected cells were treated for 24 hours, cell viability assessed using a trypan blue inclusion assay. Inset: immunoblotting showing increased level of thioredoxin in transiently transfected HeLa cells compared to empty-vector transfected cells. B. Model indicating how chaetocin competes with thioredoxin as a substrate for thioredoxin reductase, thereby serving to attenuate reduction of the thioredoxin reductase downstream substrate and effector thioredoxin. Figure numbers corresponding to experimental data supporting indicated effects are shown.

FIG. 18 (Table 1) Summary of thioredoxin reductase steady-state kinetics data for thioredoxin and tested substrates.

FIG. 19 shows that the reduction of chaetocin and thioredoxin by thioredoxin reductase displays sigmoidal kinetics. Michaelis Menton plot fitted with the Hill equation for chaetocin A. and thioredoxin B. Note the different concentration and rate scales between the chaetocin and thioredoxin plots. Data shown represent single experiments (triplicate data points; error bars, one standard deviation) representative of a minimum of three separate experiments. Error bars not evident are hidden by data points.

FIG. 20 shows mouse weight data after twice weekly 0.6 mg/kg IP administration of chaetocin.

### DETAILED DESCRIPTION

### A. Definitions

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3, or 4, solvent or water molecules.

As used herein, treatment means any manner in which one or more of the symptoms of a cancer, e.g., a hematologic or solid tumor cancer, are ameliorated or otherwise beneficially altered. Treatment also encompasses any pharmaceutical use of the compositions herein, such as uses for treating diseases, disorders, or ailments in which a cancer is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

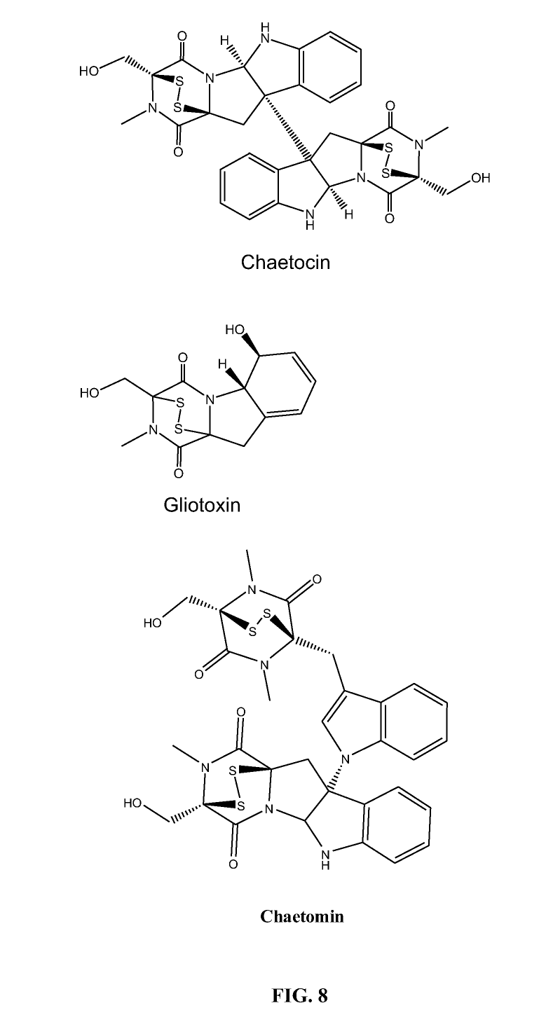

It is to be understood that the compounds provided herein may contain chiral centers. Such chiral centers may be of either the (R) or (S) configuration, or may be a mixture thereof. In certain cases, a particular configuration may be preferred, e.g., see FIG. 8 demonstrating a preferred stereochemistry for chaetocin. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” refer to carbon chains that may be straight or branched. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl).

As used herein, “cycloalkyl” refers to a saturated mono- or multi- cyclic ring system, in certain embodiments of 3 to 10 carbon atoms, in other embodiments of 3 to 6 carbon atoms. The ring systems of the cycloalkyl groups may be composed of one ring or two or more rings which may be joined together in a fused, bridged or spiro-connected fashion. Examples include, but are not limited to, cyclopropyl, cyclobutyl, cyclopentyl, and cyclohexyl.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members, where one or more, in one embodiment 1 to 4, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen.

As used herein, “carboxy” refers to a divalent radical, —C(O)O—.

As used herein, “aminocarbonyl” refers to —C(O)NH₂.

As used herein, “alkylcarbonyl” refers to —C(O)R, where R is alkyl.

As used herein, “arylcarbonyl” refers to —C(O)R, where R is aryl.

As used herein, “aminoalkyl” refers to —RNH₂, in which R is alkyl.

As used herein, “aralkyl” refers to an alkyl group that is substituted with one or more aryl groups.

As used herein, “alkaryl” refers to an aryl group that is substituted with one or more alkyl groups.

As used herein, “hydroxyalkyl” refers to an alkyl group in which one or more of the hydrogen atoms are replaced by hydroxyl (—OH).

As used herein, “alkoxy” and “alkylthio” refer to RO— and RS—, in which R is alkyl.

As used herein, “aryloxy” and “arylthio” refer to RO— and RS—, in which R is aryl.

As used herein, “amido” refers to the divalent group —C(O)NH.

As used herein, “hydrazide” refers to the divalent group —C(O)NHNH—.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. Compounds

As disclosed herein, the natural product chaetocin exhibits in vitro activity against multiple myeloma, with superior ex vivo activity and selectivity in patient myeloma cells relative to the commonly utilized anti-myeloma agents dexamethasone and doxorubicin. Additionally, chaetocin was also demonstrated to inhibit growth of myeloma in vivo. Accordingly, methods employing chaetocin and other compounds or compositions described further herein for treating or ameliorating one or more symptoms associated with multiple myeloma in a mammal, e.g., a human, are provided herein. Chaetocin or other compounds described herein can be administered either alone or in combination with other known anticancer drugs.

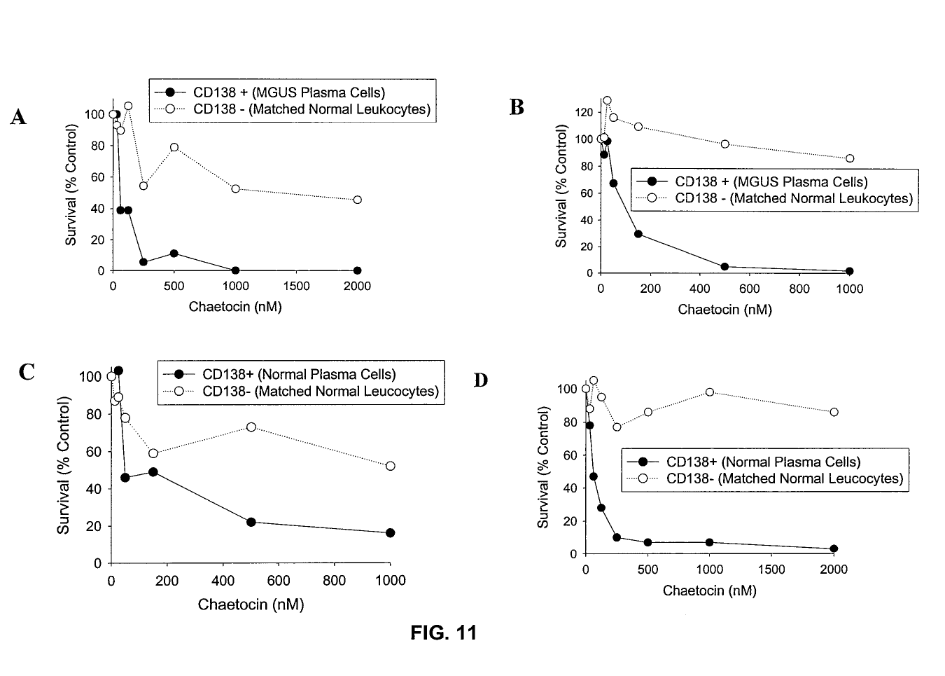

In addition, chaetocin has been shown to have activity against normal plasma cells, while largely sparing granulocytes and other leukocytes. See FIG. 11. At least one FDA-approved therapy for use in lymphomas (i.e., Rituximab) kills lymphocytes, both cancerous and noncancerous, indiscriminately. Thus, also provided herein are methods employing chaetocin and other compounds or compositions described herein for treating or ameliorating one or more symptoms associated with plasma cell B-lymphocyte lineage disorders in addition to myeloma, such as MGUS (monoclonal gammopathy of undetermined significance), amyloidosis, heavy chain diseases, cryoglobulinemia and Waldenström's macroglobulinemia.

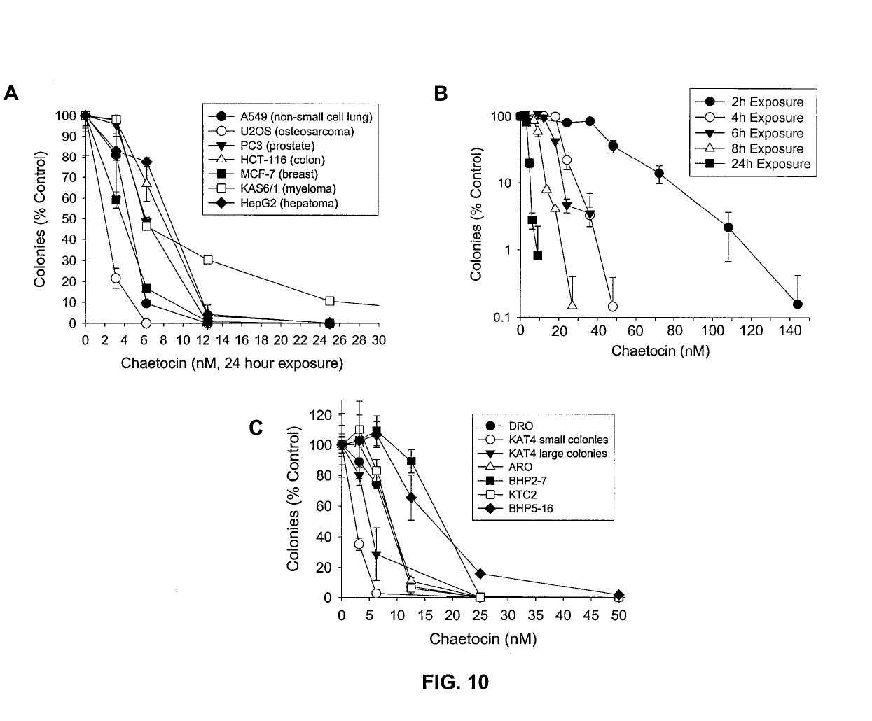

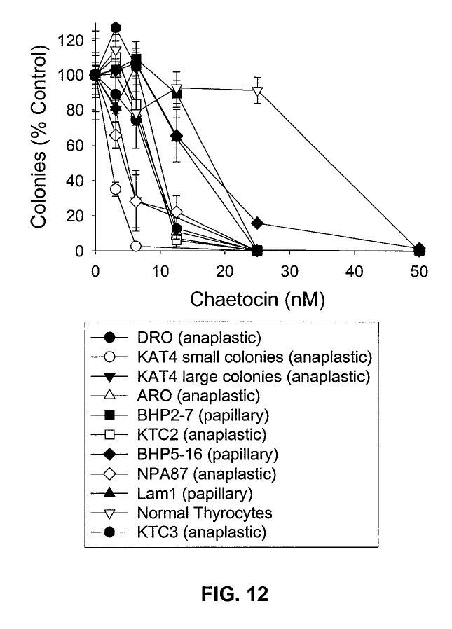

As also disclosed herein, chaetocin has broad-spectrum anti-cancer activity, including activity against solid tumors, such as lung, breast, prostate, hepatoma, thyroid and colon tumors; and sarcomas. See FIG. 10. Furthermore, chaetocin also has selectivity in killing solid tumor cancer cells. In particular, chaetocin has potent anti-cancer activity in a variety of thyroid cancer cell lines, while largely sparing normal thyrocytes (normal thyroid gland cells). See FIG. 12. Accordingly, methods for treating such solid tumors employing chaetocin and other compounds or compositions described herein are also provided.

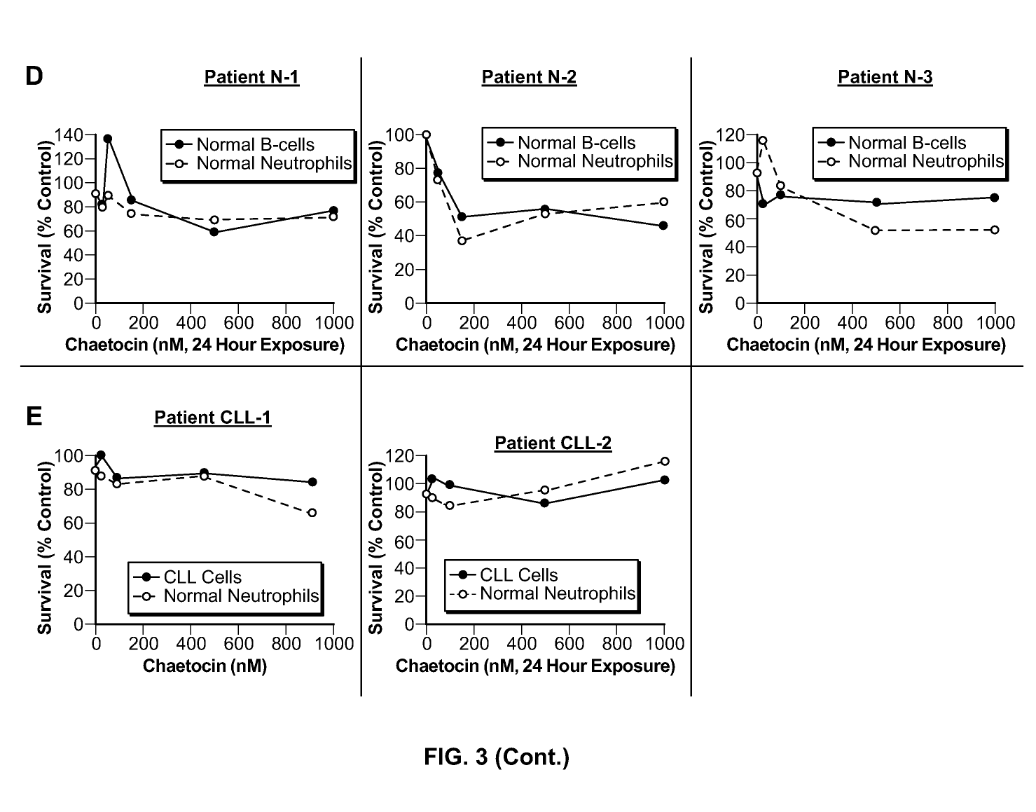

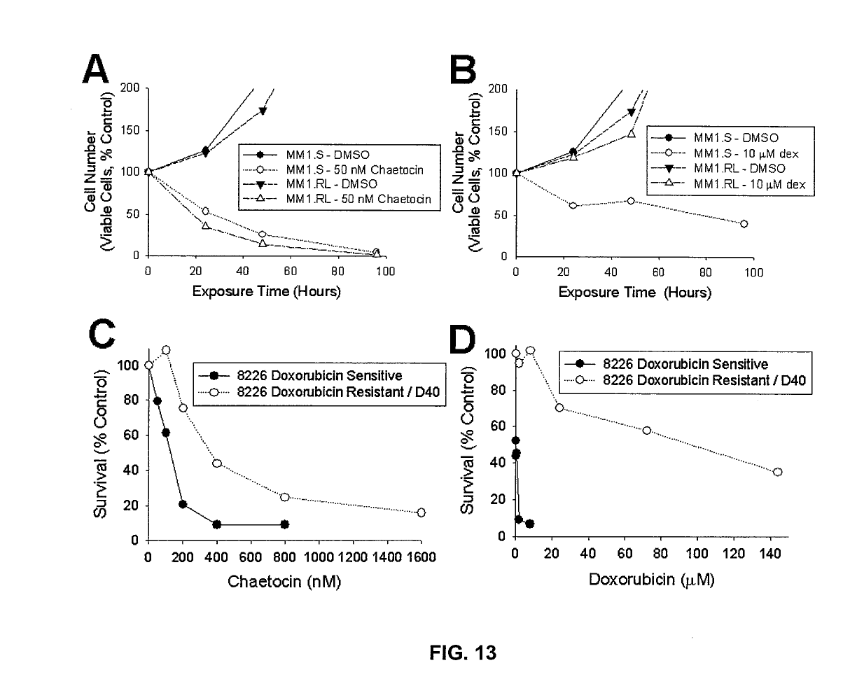

Additionally, the inventors have found that multiple myeloma cells that have acquired resistance to anti-myeloma drugs such as dexamethasone or doxorubicin are largely not cross-resistant to chaetocin (See FIG. 3A-C and FIG. 13). This suggests that chaetocin may have therapeutic application to even highly chemotherapy-resistant cancers.

Mechanistic studies of chaetocin's activity have provided insight into its anti-cancer activity. Chaetocin is rapidly accumulated in cancer cells via a transport system inhibited by glutathione and requiring intact/unreduced disulfides for uptake. Once inside the cell, its anti-cancer activity appears mediated primarily through the imposition of oxidative stress and apoptosis or cell death induction. Specifically, chaetocin has been shown to be a competitive and selective substrate for thioredoxin reductase-1 (TrxR1), an oxidative stress mitigation enzyme of central importance in limiting ROS accumulation in cancer cells, thereby linking chaetocin's enzymatic targeting to its anti-myeloma effects. This suggests that not only chaetocin, but also other agents that similarly induce cellular oxidative stress, may hold promise for further development as anti-cancer therapeutics. For example, other thiodioxopiperazine-containing natural products, such as gliotoxin and chaetomin, also are known to be cytotoxic via a mechanism of oxidative stress induction. See FIG. 8 for a comparison of the structures of gliotoxin, chaetomin, and chaetocin. Accordingly, based on such mechanistic and structural similarity, compounds related to chaeotocin, such as chaetomin and gliotoxin (e.g., compounds containing a core thiodioxopiperazine structure, and particularly containing the bridged disulfide thiodioxopiperazine structure) are proposed herein to have similar anti-cancer activities, and can be used in any of the methods described herein. Examples of such compounds include melinacidin IV, sirodesmin, hyalodendrin, sporidesmin A, leptosin, emestrin, dithiosilvatin, epicorazine, emethallicin, verticillin A and B, 19 methyl-19′deoxy-6,6′dihydroxychaetocin, aranotin, apoaranotin, and scabrosin; see also Formulas I and II below.

Use of any of the compounds provided herein, or their pharmaceutically acceptable salts or derivatives, in the treatment or amelioration of cancer is also provided, as well as use of any of the compounds, or pharmaceutically acceptable salts or derivatives, in the preparation of a medicament for the treatment or amelioration of cancer.

Compounds for use in the compositions and methods provided herein can have a structure according to Formula I:

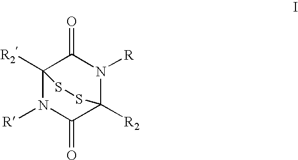

or a pharmaceutically acceptable salt or derivative thereof,

wherein R, R′, R₂, and R₂′, independently, can be selected from the group consisting of hydrogen, alkyl, cycloalkyl, alkoxy, hydroxy, aryl, heteroaryl, heteroalkyl, heterocyclyl, halo, pseudohalo, carboxy, haloalkyl, hydroxyalkyl, alkaryl, aralkyl, aminocarbonyl, aminoalkyl, alkylcarbonyl, arylcarbonyl, alkoxy, alkylthio, aryloxy, and arylthio; or

wherein R and R₂, and/or R′ and R₂′, independently, together to which the atoms to which they are attached, form a fused heterocyclic ring system which is optionally substituted with one or more alkyl, cycloalkyl, alkoxy, aryl, hydroxy, heteroaryl, heteroalkyl, heterocyclyl, halo, pseudohalo, carboxy, haloalkyl, hydroxyalkyl, alkaryl, aralkyl, aminocarbonyl, aminoalkyl, alkylcarbonyl, arylcarbonyl, alkoxy, alkylthio, aryloxy, or arylthio groups, or another optionally substituted aryl, heterocyclyl, or cycloalkyl fused ring system,

provided that R and R′ are not substituted with the same group when R₂ and R₂′ are both H, and provided that if R and R₂ form a fused ring heterocyclic ring system, then R′ and R₂′ do not also form a fused ring heterocyclic system, and if R and R₂ form a fused ring heterocyclic ring system and R′ is methyl, then R₂′ is not hydroxyalkyl or alkyl.

In some embodiments, R and/or R′ are selected from hydrogen, halo, pseudohalo, hydroxyl, alkyl having from 1 to 8 carbon atoms (e.g., 1 to 6, 1 to 5, 1 to 4, or 1 to 3 C atoms), aralkyl, where the alkyl group has from 1 to 8 carbon atoms (e.g., 1 to 6, 1 to 5, 1 to 4, or 1 to 3 C atoms), alkaryl, where the alkyl group has from 1 to 8 carbon atoms (e.g., 1 to 6, 1 to 5, 1 to 4, or 1 to 3 C atoms), hydroxyalkyl, where the alkyl group has from 1 to 8 carbon atoms (e.g., 1 to 6, 1 to 5, 1 to 4, or 1 to 3 C atoms), alkylcarbonyl, where the alkyl group has from 1 to 8 carbon atoms (e.g., 1 to 6, 1 to 5, 1 to 4, or 1 to 3 C atoms), and arylcarbonyl. In some embodiments, R and/or R′ are selected from H, methyl, ethyl, hydroxymethyl, hydroxyethyl, and hydroxypropyl.

In some embodiments, R₂ and/or R₂′ are selected from hydrogen, halo, pseudohalo, alkyl having from 1 to 8 carbon atoms (e.g., 1 to 6, 1 to 5, 1 to 4, or 1 to 3 C atoms), and hydroxyalkyl, where the alkyl group has from 1 to 8 carbon atoms (e.g., 1 to 6, 1 to 5, 1 to 4, or 1 to 3 C atoms).

In some embodiments, R₂ and/or R₂′ are hydrogen; and R and R′ are independently hydrogen, methyl, —C(O)CH₃, t-butyl, benzel, or —C(O)Ph.

### C. Preparation of the Compounds

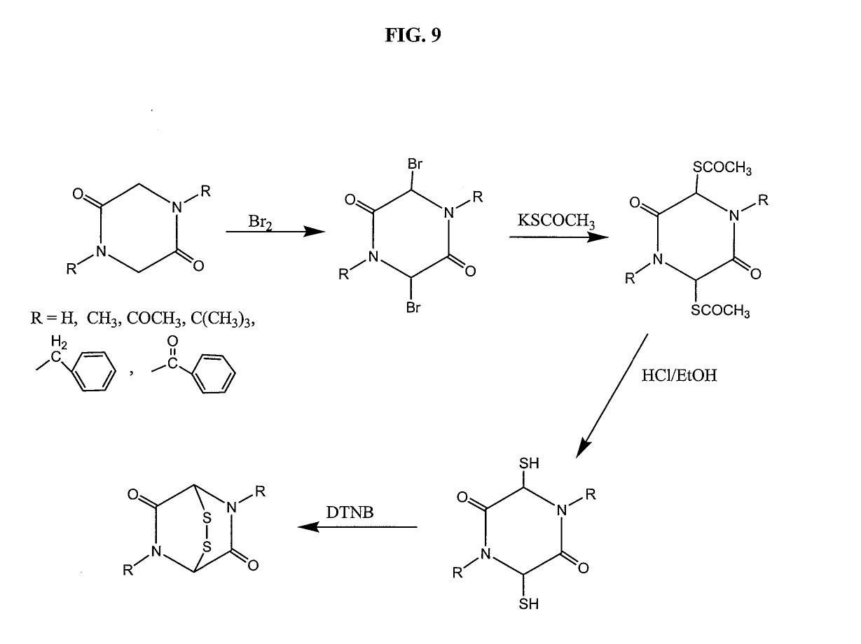

The compounds for use in the compositions and methods provided herein may be obtained from commercial sources (e.g., Sigma, St. Louis, Mo.; Aldrich Chemical Co., Milwaukee, Wis.), may be isolated from fungi using known techniques, or may be prepared by methods well known to those of skill in the art or by the methods shown herein (e.g., see FIG. 9). One of skill in the art could modify certain of the compounds using the appropriate starting materials and standard methods in organic chemistry.

### D. Formulation of Pharmaceutical Compositions

The pharmaceutical compositions provided herein contain therapeutically effective amounts of one or more of the compounds provided herein that are useful in the treatment or amelioration of one or more of the symptoms associated with a hematologic (e.g., myeloma, lymphoma, or leukemia) or solid cancers, and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats or ameliorates one or more of the symptoms of a cancer, e.g., multiple myeloma.

In one embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disorder being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), polyethylene glycol (PEG) (e.g., PEG400), cyclodextrins or cremaphor; using surfactants, such as TWEEN®, or dissolution in aqueous sodium bicarbonate. Derivatives of the compounds, such as prodrugs of the compounds or nanoparticulate formulations may also be used in formulating effective pharmaceutical compositions.

In one embodiment, a formulation can include from about 5% to about 85% PEG400, and/or from about 0.5% to about 30% DMSO. A formulation can include 0.9N NaCl, e.g., from about 15% to about 90% 0.9N NaCl. In some embodiments, a formulation can include about 15% to about 25% PEG400, about 75% to about 85% 0.9N NaCl, and from about 0.5% to about 5% DMSO. In some embodiments, DMSO is present in an amount less than 1%, e.g., 0.9%, 0.8%, 0.7%, 0.6%, 0.5%, 0.4%, 0.3%, 0.2% or 0.1%, or less.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, nanoparticles (e.g., gold, albumin) and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof, and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed or complexed with other active materials which do not impair the desired action, or with materials that supplement the desired action. The active ingredient is a compound or pharmaceutically acceptable derivative thereof as described herein. Higher concentrations, up to about 98% by weight of the active ingredient, may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

2. Injectables, Solutions, and Emulsions

Parenteral administration, in one or more embodiment characterized by injection, either subcutaneously, intramuscularly, intraperitoneally or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous, intraperitoneal and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration should be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Other embodiments may include the use of nanoparticle preparations, optionally complexed with antibodies (e.g., anti-CD138) or other substances, intended to enhance targeting to desired neoplastic cells or tissues.

7. Articles of Manufacture

The compounds or pharmaceutically acceptable derivatives may be packaged as articles of manufacture (e.g., kits) containing packaging material, a compound or pharmaceutically acceptable derivative thereof provided herein within the packaging material, and a label that indicates that the compound or composition, or pharmaceutically acceptable derivative thereof, is useful for treatment or amelioration of one or more symptoms of a cancer, including a hematologic cancer such as myeloma.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

8. Sustained Release Formulations

Also provided are sustained release formulations to deliver the compounds to the desired target at high circulating levels (between 10⁻⁹ and 10⁻⁴ M). The levels are either circulating in the patient systemically, or in one embodiment, localized to a site of, e.g., paralysis.

It is understood that the compound levels are maintained over a certain period of time as is desired and can be easily determined by one skilled in the art. Such sustained and/or timed release formulations may be made by sustained release means of delivery devices that are well known to those of ordinary skill in the art, such as those described in U.S. Pat. Nos. 3,845,770; 3,916,899; 3,536,809; 3, 598,123; 4,008,719; 4,710,384; 5,674,533; 5,059,595; 5,591,767; 5,120,548; 5,073,543; 5,639,476; 5,354,556 and 5,733,566, the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions provided herein. Thus, single unit dosage forms suitable for oral administration, such as, but not limited to, tablets, capsules, gelcaps, caplets, powders and the like, that are adapted for sustained release are contemplated herein.

In one embodiment, the sustained release formulation contains active compound such as, but not limited to, microcrystalline cellulose, maltodextrin, ethylcellulose, and magnesium stearate. As described above, all known methods for encapsulation which are compatible with properties of the disclosed compounds are contemplated herein. The sustained release formulation is encapsulated by coating particles or granules of the pharmaceutical compositions provided herein with varying thickness of slowly soluble polymers or by microencapsulation. In one embodiment, the sustained release formulation is encapsulated with a coating material of varying thickness (e.g., about 1 micron to 200 microns) that allow the dissolution of the pharmaceutical composition about 48 hours to about 72 hours after administration to a mammal. In another embodiment, the coating material is a food-approved additive.

In another embodiment, the sustained release formulation is a matrix dissolution device that is prepared by compressing the drug with a slowly soluble polymer carrier into a tablet. In one embodiment, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, having a relatively high percentage of plasticizer in the coating in order to permit sufficient flexibility to prevent substantial breakage during compression are disclosed. The specific amount of plasticizer varies depending on the nature of the coating and the particular plasticizer used. The amount may be readily determined empirically by testing the release characteristics of the tablets formed. If the medicament is released too quickly, then more plasticizer is used. Release characteristics are also a function of the thickness of the coating. When substantial amounts of plasticizer are used, the sustained release capacity of the coating diminishes. Thus, the thickness of the coating may be increased slightly to make up for an increase in the amount of plasticizer. Generally, the plasticizer in such an embodiment will be present in an amount of about 15 to 30% of the sustained release material in the coating, in one embodiment 20 to 25%, and the amount of coating will be from 10 to 25% of the weight of the active material, and in another embodiment, 15 to 20% of the weight of active material. Any conventional pharmaceutically acceptable plasticizer may be incorporated into the coating.

The compounds provided herein can be formulated as a sustained and/or timed release formulation. All sustained release pharmaceutical products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: 1) extended activity of the composition, 2) reduced dosage frequency, and 3) increased patient compliance. In addition, sustained release formulations can be used to affect the time of onset of action or other characteristics, such as blood levels of the composition, and thus can affect the occurrence of side effects.

The sustained release formulations provided herein are designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds.

Preparations for oral administration may be suitably formulated to give controlled release of the active compound. In one embodiment, the compounds are formulated as controlled release powders of discrete microparticles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to those described in column 3, line 46-column 4, line 8 of U.S. Pat. No. 5,354,556 which is incorporated by reference in its entirety.

The sustained release compositions provided herein may be formulated for parenteral administration, e.g., by intramuscular injections or implants for subcutaneous tissues and various body cavities and transdermal devices. In one embodiment, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil suspensions and solutions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachis, maize, almond, soybean, cottonseed and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

### E. Evaluation of the Activity of the Compounds

The activity of the compounds provided herein for anti-cancer activity and/or cell selectivity, may be measured in standard assays, e.g., in vitro cell proliferation or cell death, apoptosis, cellular uptake, and oxidative stress assays, including those described in the Examples herein.

### F. Methods of Use of the Compounds and Compositions

Provided herein are methods to treat or ameliorate symptoms or disorders associated with a cancer, including hematologic and solid tumor cancers. The methods include administering one or more of the compounds described herein, or a pharmaceutically acceptable salt or derivative thereof, or a composition or pharmaceutical composition comprising the same, to a mammal, e.g., a human, cat, dog, horse, pig, cow, sheep, mouse, rat, or monkey. In some embodiments, the compound is one according to Formula I, as described above. In some cases, the compound administered is chaetocin, or a pharmaceutically acceptable salt or derivative thereof, having the structure as set forth in FIG. 8. In other embodiments, the compound administered is chaetomin or gliotoxin, or a pharmaceutically acceptable salt or derivative thereof, having the structures as set forth in FIG. 8. In yet other embodiments, the compound administered comprises a bridged disulfide thiodioxopiperazine ring. In yet other embodiments, the compound administered is selected from melinacidin IV, sirodesmin, hyalodendrin, sporidesmin A, leptosin, emestrin, dithiosilvatin, epicorazine, emethallicin, verticillin A and B, 19 methyl-19′deoxy-6,6′dihydroxychaetocin, aranotin, apoaranotin, and scabrosin, or a pharmaceutically acceptable salt or derivative thereof.

In some cases, a method for treating or ameliorating a cancer can include administering to a mammal a compound according to Formula II:

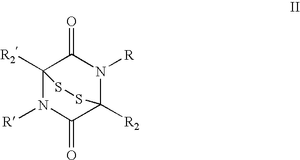

or a pharmaceutically acceptable salt or derivative thereof,

wherein R, R′, R₂, and R₂′, independently, can be selected from the group consisting of hydrogen, alkyl, cycloalkyl, alkoxy, hydroxy, aryl, heteroaryl, heteroalkyl, heterocyclyl, halo, pseudohalo, carboxy, haloalkyl, hydroxyalkyl, alkaryl, aralkyl, aminocarbonyl, aminoalkyl, alkylcarbonyl, arylcarbonyl, alkoxy, alkylthio, aryloxy, and arylthio; or

wherein R and R₂, and/or R′ and R₂′, independently, together to which the atoms to which they are attached, form a fused heterocyclic ring system which is optionally substituted with one or more alkyl, cycloalkyl, alkoxy, aryl, hydroxy, heteroaryl, heteroalkyl, heterocyclyl, halo, pseudohalo, carboxy, haloalkyl, hydroxyalkyl, alkaryl, aralkyl, aminocarbonyl, aminoalkyl, alkylcarbonyl, arylcarbonyl, alkoxy, alkylthio, aryloxy, or arylthio groups, or another optionally substituted aryl, heterocyclyl, or cycloalkyl fused ring system,

and provided that if R and R₂ form a fused ring heterocyclic ring system, then R′ and R₂′ do not also form a fused ring heterocyclic system, and if R and R₂ form a fused ring heterocyclic ring system and R′ is methyl, then R₂′ is not hydroxyalkyl or alkyl.

In some embodiments, the cancer can be multiple myeloma. In other embodiments, the cancer can be another B-lymphocyte lineage disorder, such as MGUS, amyloidosis or a leukemia or lymphoma. In yet other cases, the cancer can be a solid tumor. In certain embodiments, the symptoms or disorders associated with multiple myeloma include one or more of the following: production of monoclonal proteins in serum and/or urine, destructive boney lesions, renal failure, hypercalcemia, loss of appetite, fatigue, muscle weakness, restlessness, difficulty in thinking or confusion, constipation, increased thirst, increased urine production, nausea and vomiting, pain, e.g., in the lower back and ribs, anemia, primary and recurrent infections, neuropathy, pneumonia, and hyperviscosity of the blood.

In practicing the methods, effective amounts of the compounds or composition provided herein are administered. Such amounts are sufficient to achieve a therapeutically effective concentration of the compound or active component of the composition in vivo.

Any of the compounds or compositions can also be used for inducing oxidative stress in a cell, e.g., in a cancerous cell. In the method, a cell can be exposed to or contacted with a compound or composition described herein to induce oxidative stress. Exposure or contact can be in vivo or in vitro. Oxidative stress can be monitored using known assays, including fluorescent (e.g. FACS) assays, e.g., as described below. In addition, any of the compounds or compositions can be used to induce cell death or apoptosis in a cell, e.g., a cancerous cell; apoptosis/cell death can be monitored as described herein or by standard assays known to those having ordinary skill in the art.

### Examples

### Materials and Methods

Reagents: Chaetocin, doxorubicin, dexamethasone, apicidin, trichostatin A, reduced glutathione, N-acetyl cysteine, H₂O₂, aphidicolin, DRB (5,6-dichloro-1-beta-D-ribofuranosylbenzimidazole), and cycloheximide were purchased from Sigma (St. Louis, Mo.); and tetramethylrhodamine methyl ester (TMRM) from Invitrogen (Carlsbad, Calif.). LAQ-824 (a small molecule HDAC inhibitor currently in clinical trials) was kindly provided by Drs. Chunrong Yu and Alex Adjei (Mayo Clinic, Rochester, Minn.).

Cell culture: Cells were cultured in the following media: A549 (obtained from ATCC, Chicago Ill.) in RPMI 1640 containing 5% (v/v) FBS; myeloma cell lines KAS6/1 OCI-MY5, MM1S/4 and RPMI 8226 in RPMI 1640 containing 10% FBS; patient bone marrow cells in MEM containing 20% FBS; KAS6/1 cells were supplemented with 1 ng/mL IL-6. All media contained 100 U/mL penicillin G, 100 μg/mL streptomycin and 2 mM 1-glutamine. Cells lines were passaged twice weekly and maintained at 37° C. in an atmosphere containing 95% air-5% CO₂ (v/v).

Patient samples: All described ex vivo experiments utilized excess/waste bone marrow cells obtained from patients in conjunction with their standard clinical care and in accord with IRB-approved protocols.

Colony formatting assays: Briefly, 650 suspended A549 cells obtained from trypsinization of stock flasks of subconfluent cell cultures were deposited into each of triplicate sets of 35 mm tissue culture plates and allowed to adhere for 16 hours. Cells were then treated for the indicated durations (24 hours unless otherwise indicated) with diluent and/or drugs as indicated in the text and Figures. After drug removal and washing, cells were allowed to proliferate in drug-free medium for 7 to 10 days, and thereafter washed twice with serum-free phosphate buffered saline (PBS), stained with Coomassie blue, and then manually counted.

Assessment of drug effects on patient cells: Patient bone marrow cells were collected via posterior superior iliac crest bone marrow aspiration under local anesthesia in accord with approved Mayo Clinic IRB protocols. Patient bone marrow leukocytes were divided into myeloma (CD138+) and non-myeloma/normal leukocyte (CD138−) fractions employing sorting using magnetic bead technology in kit form (MACS CD138 microbeads, Miltenyi Biotech, Auburn, Calif.). Sorted cells were plated in a 96-well tissue culture plate at a concentration of 5×10⁵ cells and dosed with indicated drug concentrations for 24 hours. Survival was assessed using a trypan blue exclusion assay.

FACS analyses assessing the effects of chaetocin on marrow cell populations was accomplished using annexin V (Caltag Laboratories, Burlingame, Calif.) and 7 amino-actinomycin D (7-AAD, Calbiochem, San Diego, Calif.) staining to identify viable, apoptotic and dead cells. Briefly, 100 μl of treated unsorted cells washed and resuspended in annexin binding buffer (ABB, 0.15 M NaCl, 0.0033 M CaCl₂ HEPES buffer, pH 7.4; 1×10⁶ cells/100 μL) was added to each of two tubes along with 5 μL each anti-annexin V fitc, anti-CD38 pe (phycoerythrin), and anti-CD45 apc in one tube; and anti-CD56 fitc, anti-CD38 pe, and anti-CD45 apc in the second. Cells were incubated (4° C., 15 min), washed with ABB, resuspended in 500 μL of ABB; and 2.5 μL of 7-AAD (2 mg/mL stock) was then added to each tube. Cells were subsequently incubated 15 minutes, washed, resuspended in 500 μL of ABB, and immediately run on FACScaliber (BD Biosciences, San Jose, Calif.) with data analyzed using Cellquest Pro Software (BD Biosciences, San Jose, Calif.). Multivariate analysis using the typical CD45−, bright CD38 gating and FSC vs. SSC (size vs. granularity) identified the viable, apoptotic and dead fractions of examined cell populations.

B-cell chronic lymphocytic leukemia (B-CLL) cells were obtained from patients with persistent lymphocytosis of >5000 lymphocytes/mm³ and a CD5+, dim surface Ig expression and monoclonal κ or λ expression. Peripheral blood mononuclear cells (PBMCs) isolated from heparinized blood by Ficoll (Gallard-Schlesinger Industries, Inc., Plainview, N.Y.) density gradient centrifugation were washed twice with normal saline, counted using a Vi Cell XR cell viability analyzer (Beckman Coulter, Fullerton, Calif.), resuspended to 100 million/mL in PBS with 2% FBS; and the B-cell population was isolated using the Human B Cell Enrichment Kit (without CD43 depletion; StemCell Technologies, Vancouver, BC, Canada) in conjunction with a RoboSep Fully Automated Cell Separator (StemCell Technologies). After separation, cells were washed twice in sterile saline and counted. Cell purity assessed with CD19 FITC and CD5 PE (both from BD Biosciences, San Jose, Calif.) was routinely above 98%. Resulting red blood/polynuclear pellets from the Ficoll step were lysed by addition of 5 ml ACK Lysis Solution (BioSource International/Invitrogen, Carlsbad, Calif.; 10 minutes, 37° C.), washed twice with sterile saline and used for subsequent neutrophil experiments. Isolation of normal human B-cells utilized a similar procedure, saving that Human CD19 Positive Selection Kit (StemCell Technologies) was used for separation on the RoboSep described above.

Assessment of apoptosis: Apoptosis was assessed using transmission electron microscopy and Hoechst 33258 staining using fluorescence microscopy by examining cells for apoptotic morphological changes, expressing number of apoptotic cells at a percentage of 200 total counted cells, as previously described (Bible K C and Kaufmann S H, Flavopiridol (NSC 649890, L86-8275): A cytotoxic flavone that induces death in non-cycling A549 human lung carcinoma cells. Cancer Res. 1996; 56:4856-4861).

Evaluation of loss of mitochondrial membrane potential (ΔΨₘ): Chaetocin-treated OCI-MY5 cells were sedimented, washed with PBS, resuspended in medium, stained with TMRM (50 nM, 45 minutes, 37° C.), placed on ice for 5 minutes and immediately subjected to flow microfluorometry using a FACScan flow cytometer (Becton Dickinson, Mountain View, Calif., USA; 488 nm laser). Fluorescence emission was observed through a 585/42 nm filter, and 20,000 events were analyzed using CellQuest software (Verity Software House, Topsham, Me., USA).

Immunoblotting: Cells grown in suspension culture at densities of 3-6×10⁵ cells/mL and treated as indicated were washed three times with PBS, lysed in lysis buffer (50 mM Tris-HCl pH 8.0, 400 mM NaCl, 0.5% NP-40, 10% glycerol, 1 mM EDTA supplemented immediately before use with 10 ng/μL pepstatin A, 500 μM PMSF, 10 ng/μL leupeptin, 10 ng/μL aprotinin, and 200 μM sodium orthovanidate), and processed for SDS-polyacrylamide gel electrophoresis and subsequent immunoblotting for acetylated histone H3 (Cell Signaling Technology, Beverly, Mass.), poly ADP-ribose polymerase (PARP; BD PharMingen, San Diego, Calif.) and actin (Sigma, St. Louis, Mo.).

Evaluation of DNA ladder formation: Chaetocin- and diluent-treated OCI-MY5 cells treated as indicated were washed twice with PBS, and processed for DNA electrophoresis; with gel bands visualized and documented using a Syngene Image documentation system (Frederick, Md.).

Measurement of intra- and extracellular chaetocin levels: Tumor cells treated with indicated chaetocin concentrations were washed twice with ice cold PBS, immediately solubilized in 0.5 M perchloric acid, and evaluated “real time” via HPLC using Beckman System Gold Nouveau Software with a dual pump 125 gradient pump system, 507e autosampler, 168 diode array detector, and Beckman Ultrasphere ODS column (4.6 mm×15 mm×7 μm) using the following elution profile: 100% water to 100% methanol linear gradient over 40 minutes, followed by a 10 minute period of elution with 100% methanol. Quantitation was accomplished using a standard curve of peak areas derived from analogous HPLC data using varying concentrations of chaetocin.

Chemical modifications of chaetocin: Reduced chaetocin was prepared by treating chaetocin with 100 mM dithiothreitol for 30 minutes at room temperature. To synthesize S-methyl chaetocin, sodium borohydride (1.7 mg, 0.046 mmol) was added to chaetocin (2 mg, 0.0029 mmol) in 75% dichloromethane and 25% methanol at 0° C. under dry nitrogen and stirred for 1 hour before addition of methyl iodide; see Isham et al., Blood 109:2579-2587 (2007). This solution was stirred for an additional 16 hours, washed with 10% HCl (1×0.5 mL), and the organic layer was extracted with dichloromethane (3×1 mL). The organic phases were combined, dried over magnesium sulfate, and evaporated to dryness. When compared to the ¹H NMR spectra of chaetocin, two new peaks were observed (2.2 ppm, s, 6H, 1.8 ppm, s, 6H), confirming reduction and methylation of the disulfide bonds. LC-MS was also performed to confirm the identity of the compound: Calculated: [M⁺]=757.8. Found: [M⁺]=757.2, [M+NH₄⁺]=774.2, [M+Na⁺]=779.2, [M+K⁺]=795.1.

Assessment of cellular oxidative stress: Cellular oxidative stress was assessed utilizing 5,6-chloromethyl-2′,7′-dichlorodihydrofluorescein diacetate or hydroethidium (Molecular Probes/Invitrogen, Carlsbad, Calif.) as cell permeable fluorescent probes and FACS analyses. Briefly, cells were treated with the indicated drug for 24 hours, incubated in media containing 5.4 μg/ml probe at 37° C. for 15 minutes, sedimented and resuspended in PBS before flow microfluorimetry (FACScan flow cytometer; Becton Dickinson, Mountain View, Calif.) with a 488 nm laser. Fluorescence emission was observed through a 530/30 nm filter, and 20,000 events were analyzed using CellQuest software (Verity Software House, Topsham, Me.).

Assessment of intracellular glutathione: Reduced glutathione was quantitated utilizing a kit (Cayman Chemical; Ann Arbor, Mich.) that employs glutathione reductase and colorimetric detection of the breakdown product of DTNB. Briefly, treated cells were washed, lysed by sonication in MES buffer, and lysates were deproteinated by metaphosphoric acid. The assay quantitated oxidized glutathione alone, using 2-vinylpyridine to derivatize GSH, and total glutathione (detection at 405 nm; Beckman AD340 plate reader, Beckman Coulter, Fullerton, Calif.). Reduced glutathione values were obtained by subtracting oxidized from total glutathione.

Statistics: Differences between cell lines were assessed using two-sided t tests and pooled estimates of variance.

In vivo experiments: The in vivo effects of chaetocin were preliminarily explored at Southern Research Institute with assistance from Dr. William Waud (Birmingham, Ala.) in SCID mice using subcutaneously flank-implanted RPMI 8226 myeloma xenografts using a twice-weekly intraperitoneal administration schedule, with chaetocin formulated in 25% DMSO and 75% PEG400. Tumor weights were calculated from volumes derived from direct tumor measurements. The control group consisted of 10 animals, whereas each treatment group consisted of 6 animals each.

Subsequent in vivo work in mice utilized the following formulation: 0.06 mg/mL chaetocin in 20% PEG400 and 79.14% 0.9N NaCl with 0.86% DMSO as a cosolvent. This system retains in vitro activity in cell culture models.

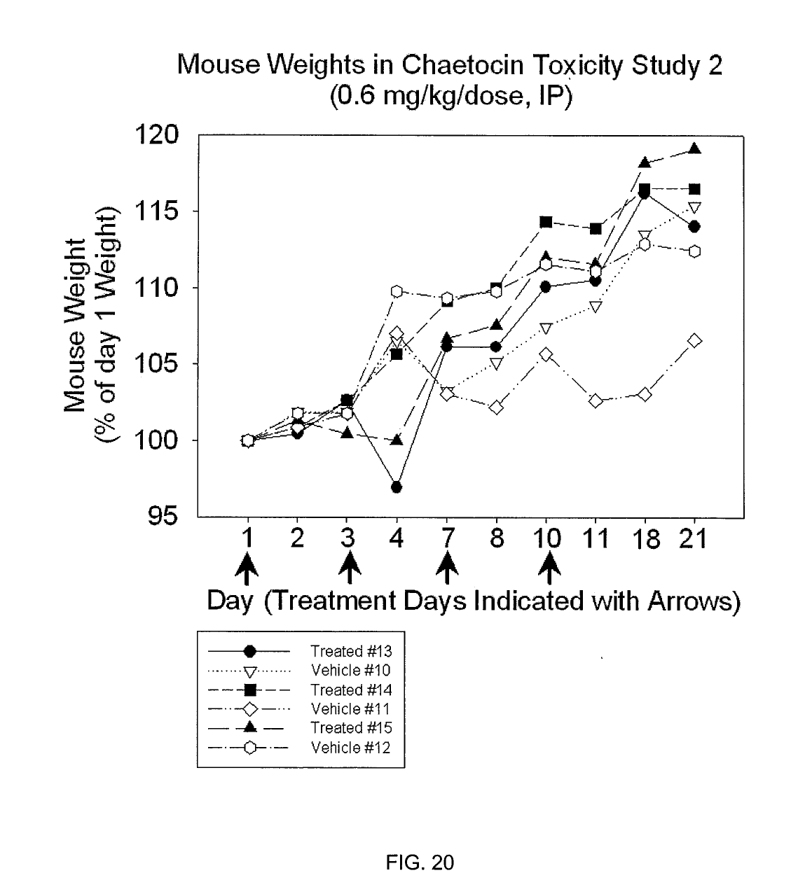

Both 0.6 mg/kg and 0.25 mg/kg chaetocin given IP were tolerated in mice treated twice weekly for two weeks, although some GI toxicity was noted. Mouse weights after treatment with chaetocin are indicated in FIG. 20.

### Materials and Methods for Thioredoxin Reductase Experiments

Reagents: Chaetocin, β-Nicotinamide adenine dinucleotide 2′-phosphate reduced tetrasodium salt hydrate (NADPH), rat liver thioredoxin reductase, 5,5′-dithiobis(2-nitrobenzoic acid) (DTNB), oxidized glutathione (GSSG), bovine insulin, gliotoxin, thioredoxin reductase assay kit, anti-actin antibody and Celytic lysis reagent were purchased from Sigma (St. Louis, Mo.); yeast glutathione reductase and Complete Protease Inhibitor Tablets from Roche (Indianapolis, Ind.); BCA protein from Pierce (Rockford, Ill.); oxidized E. coli thioredoxin and dithiothreitol (DTT) from Promega (Madison, Wis.); verruculogen from Calbiochem (San Diego, Calif.); 4-acetamido-4′-maleimidylstilbene-2,2′-disulfonic acid (AMS) and Lipofectamine-Plus from Invitrogen (Carlsbad, Calif.); chaetomin from Alexis Biochemicals (San Diego, Calif.); and anti-thioredoxin antibody from Cell Signaling (Beverly, Mass.). HeLa cells were obtained from ATCC.

Thioredoxin Reductase Activity Assay (DTNB method): Cell-free thioredoxin reductase activity was assayed in 100 mM potassium phosphate (pH 7.0), 10 mM EDTA according to the Sigma kit protocol. Final concentrations were 0.0005 U/μL of enzyme and 0.24 mM NADPH in the presence of chaetocin as indicated in a 100 μL reaction. The reaction was started by the addition of DTNB (3 mM) and the change in absorbance at 405 nm was monitored in a plate reader. Activity was calculated as the increase in absorbance between 2 and 5 min after DTNB addition.

Glutathione Reductase Activity Assay: Cell-free glutathione reductase activity was assayed in 100 mM potassium phosphate (pH 7.0), 10 mM EDTA. The 200 μL reaction mixture comprised 0.00006 U/μL of enzyme, 0.75 mM DTNB, 0.1 mM NADPH and varying concentrations of chaetocin as indicated. The reaction was started by addition of oxidized glutathione (1 mM) and was monitored in a plate reader at 405 nm. Activity was calculated as the increase in absorbance between 1 and 3 minutes after glutathione addition.

Thioredoxin Reductase Activity Assay (gel-based oxidation state of thioredoxin method): Reduction of thioredoxin by thioredoxin reductase was measured by the decrease in electrophoretic mobility caused by covalent modification of thioredoxin (by AMS) when the disulfide is reduced. The reaction mix contained 100 mM potassium phosphate (pH 7.0), 10 mM EDTA, 0.24 mM NADPH, chaetocin or other compounds as indicated, 50 μM oxidized thioredoxin and 0.0002 U/μL thioredoxin reductase (except for the initial rate Kᵢ experiment, which contained 0.00005 U/μL thioredoxin reductase). At the indicated time, a 5 μL sample was removed and immediately added to 5 μL of 30 mM AMS in TE buffer (pH 7.5). The AMS was allowed to react (15 minutes at 22° C.) with reduced thioredoxin sulfhydryl groups, then the samples were mixed with non-reducing sample buffer and were electrophoresed on 18% Tris-HCl SDS-PAGE gels. The gels were stained with Coomassie blue and bands were imaged and quantitated using a Syngene InGenius gel documentation system (Frederick, Md.).

Thioredoxin Activity Assay: The 100 μL reaction contained 100 mM potassium phosphate (pH 7.0), 2 mM EDTA, 0.13 mM bovine insulin, 3.9 μM E. coli thioredoxin and chaetocin as indicated. The reaction was initiated by addition of 0.33 mM DTT, and turbidity was monitored at 620 nm in a plate reader. The initial linear rate was calculated based on the slope of the line after an increase in absorbance (indicating precipitation of insulin) started to occur.

Steady-state kinetics: The 100 μL reaction consisted of 100 mM potassium phosphate (pH 7.0), 10 mM EDTA, 0.0004 U/μL thioredoxin reductase and chaetocin or other compounds as indicated. The assay was carried out in a 96-well quartz plate and the oxidation of NADPH was measured as a change in absorption at 340 nm. The initial velocities of the reaction were calculated from the decrease in the absorbance between 0 and 5 min (slope), and on a pathlength of 0.3 cm and NADPH ε=6.22 mmol⁻¹*cm⁻¹*L. The velocity versus concentration data were then analyzed using SigmaPlot's Enzyme Kinetics 1.3 program (Systat Software, San Jose, Calif.).

Synthesis of S-methylchaetocin: Chaetocin was reduced with sodium borohydride and methylated using methyl iodide as previously described.

Mass spectrometry: Three samples were analyzed: “chaetocin”=2.04 μg chaetocin in 100 mM potassium phosphate (pH 7.0), 10 mM EDTA; “chaetocin+NADPH”=2.04 μg chaetocin in 100 mM potassium phosphate (pH 7.0), 10 mM EDTA with 0.24 mM NADPH; and “chaetocin+NADPH+TrxR1”=2.04 μg chaetocin in 100 mM potassium phosphate (pH 7.0), 10 mM EDTA with 0.24 mM NADPH and 2.04 μg thioredoxin reductase. Samples were allowed to react at 22° C. for 15 min and then processed for analysis by desalting and fractionating using C18 ZipTips (Millipore Corporation, Bedford, Mass.). For the DTT/chaetocin reaction, the reaction proceeded for 20 min and was then spun down to remove precipitated insulin before loading onto ZipTips. ZipTips were conditioned with 60% acetonitrile:39% water:1% acetic acid and equilibrated with 1% acetic acid. Samples were acidified with glacial acetic acid and loaded onto an equilibrated ZipTip and washed with 1% acetic acid. Fractions were step-eluted off the ZipTip using 10 μl of 1% acetic acid in water containing 20, 30, 40, or 60% acetonitrile. Collected fractions were injected by loop injection (2 μl) directly into the mass spectrometer using a mobile phase of 30% acetonitrile:69% water:1% acetic acid at 5 μl/min. Mass spectra were collected on an Agilent Technologies LC/MSD-TOF mass spectrometer in positive electrospray ionization mode over a m/z range of 400 to 1500. The capillary, fragmentor, skimmer, and OCT RF voltages (3500, 185, 60, 200 volts, respectively) were optimized to enhance signal and minimize instrument fragmentation.

Cell culture, transient transfections, and immunoblotting: HeLa cells were cultured in RPMI 1640 containing 5% FBS and 2 mM L-glutamine. Cells were passaged twice weekly and maintained in 37° C. in an atmosphere containing 95% air-5% CO₂ (vol/vol). For transfections, 10⁵ cells per well were plated in 12-well plates and were transfected with 1 μg pcDNA or pcDNA-Trx using standard Lipofectamine-Plus procedures. Transfection efficiency based on cells transfected with GFP was 80%. Twenty four hours after transfection the cells were treated with either DMSO, 100 nM chaetocin or 100 nM doxorubicin for 24 hours. The cells were then trypsinized and manually counted in trypan blue to exclude dead cells. The treatments were done in triplicate and the experiment was repeated three times. For immunoblotting (24 hours after transfections), cells were trypsinized, washed in cold PBS, and lysed in Celytic lysis buffer containing Complete protease inhibitors. Protein was analyzed by BCA assay and lysates were electrophoresed on 15% SDS-PAGE gels and transferred to nitrocellulose. Immunoblotting for thioredoxin and actin was then performed.

Statistics: Statistical significance was assessed using pooled estimates of variance and the two-sided T-distribution.

### Results

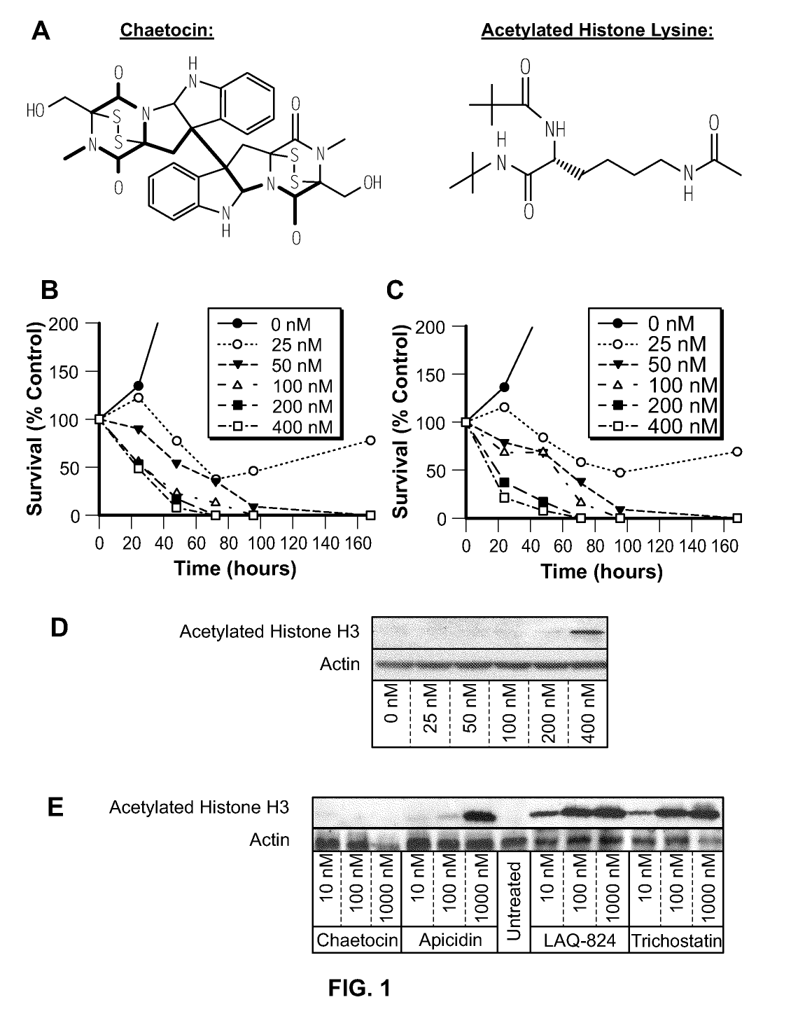

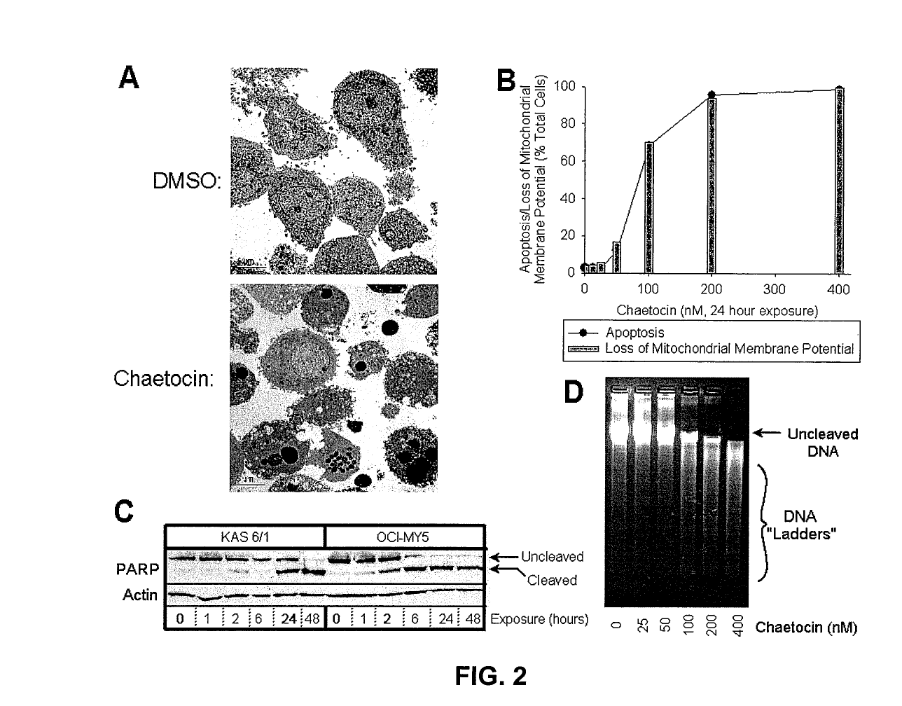

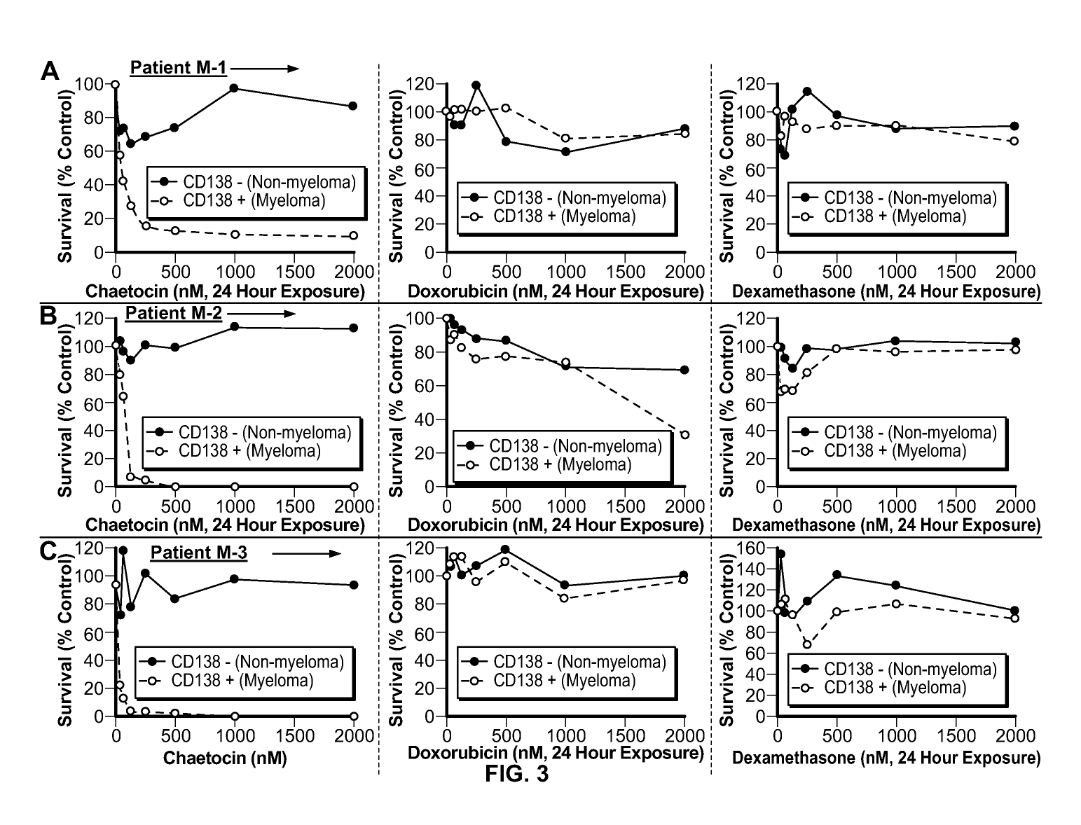

1. Chaetocin potently kills IL-6 dependent and independent myeloma cell lines. To preliminarily assess the anti-myeloma effects of chaetocin, IL-6-dependent KAS-6, and IL-6-independent OCI-MY5 cells were utilized. Chaetocin (FIG. 1A) readily killed both lines without apparent cellular recovery at chaetocin concentrations above about 25 nM (FIGS. 1B and C). Other tested myeloma cell lines including U266 and MM1 were generally also similarly facilely killed by chaetocin (data not shown).
2. Chaetocin has structural similarities to histone deacetylase inhibitors (HDACIs) yet does not appreciably alter cellular levels of acetylated histone H3 at cytotoxic concentrations. As chaetocin bears a high degree of structural similarity to the acetylated histone lysine moiety mimicked by many HDACIs ((Colletti S L, Myers R W, Darkin-Rattray S J, et al., Broad spectrum antiprotozoal agents that inhibit histone deacetylase: structure-activity relationships of apicidin, Part 1: Bioorg Med Chem. Lett. 2001; 11:107-11); FIG. 1A), HDAC inhibition accompanying chaetocin-induced cytotoxicity was probed. Although treatment of A549 human small cell lung cancer cells with chaetocin produced increased acetylated histone H3 at high nanomolar concentrations (FIG. 1D), treatment of U266, KAS 6/1 or OCI-MY5 human myeloma cells had no effects on cellular levels of acetylated histone H3 (FIG. 1E and data not shown). Hence, although chaetocin-induced HDAC inhibition was observed in some cell lines, it was not observed in tested myeloma lines at cytotoxic concentrations.
3. Chaetocin kills myeloma cells in vitro via induction of morphological apoptosis accompanied by DNA ladder formation and PARP cleavage. In vitro anti-myeloma activity of chaetocin was associated with induction of apoptotic morphological changes as assessed by electron microscopy (FIG. 2A), Hoechst 33258 staining (FIG. 2B), loss of mitochondrial membrane potential (FIG. 2B), PARP cleavage (FIG. 2C), and DNA ladder formation (FIG. 2D).
4. Chaetocin selectively kills freshly collected sorted patient CD138+ myeloma cells with superior efficacy to the commonly utilized anti-myeloma agents dexamethasone and doxorubicin. Rigorous studies were undertaken employing freshly collected sorted CD 138+ myeloma cells from 12 patients in accord with an approved IRB protocol, using matched negatively sorted patient CD138− bone marrow leukocytes as controls. In all evaluated patient samples, chaetocin demonstrated dramatic anti-myeloma activity while largely sparing matched (CD138−) normal bone marrow leukocytes (FIG. 3A-C; representative results from three patients shown). Impressively, the anti-myeloma effects of chaetocin were uniformly superior to those produced by the first-line anti-myeloma agents doxorubicin and dexamethasone under identical conditions (FIG. 3A-C). Furthermore, the potent and selective anti-myeloma effects of chaetocin were observed in samples obtained from patients afflicted with a broad range of different myeloma subtypes, including smoldering myeloma and heavily treated myeloma post-peripheral blood stem cell transplantation; as well as in myeloma cells demonstrating a wide array of cytogenetic abnormalities.

To preliminarily explore whether the observed superior ex vivo activity of chaetocin in comparison to dexamethasone or doxorubicin might reflect the absence of cross-resistance to chaetocin and these other agents, preliminary studies using paired dexamethasone-sensitive and resistant or doxorubicin-sensitive and resistant myeloma cell lines were undertaken. The MM1S and MM1R lines were used to evaluate cross resistance to dexamethasone, to find that dexamethasone-resistant MM1R cells were as sensitive to chaetocin-induced cytotoxicity as were dexamethasone-sensitive MM1S cells (data not shown). Hence, chaetocin appears not to be cross resistant to dexamethasone-resistant myeloma cell lines or patient samples. In contrast, when RPMI 8226 paired doxorubicin-sensitive and doxorubicin-resistant myeloma cells were examined, there was moderate cross resistance to chaetocin (data not shown). Because the mechanism involved in doxorubicin-resistance in the utilized RPMI 8226 cells is known to primarily involve overexpression of P-glycoprotein (PgP), these data suggest that chaetocin, like doxorubicin, may be a substrate for PgP. Of note is that none of the patients from which myeloma cells were obtained for ex vivo chaetocin sensitivity testing had been previously treated with doxorubicin, perhaps accounting for the fact that chaetocin resistance was not observed in patient myeloma cells.

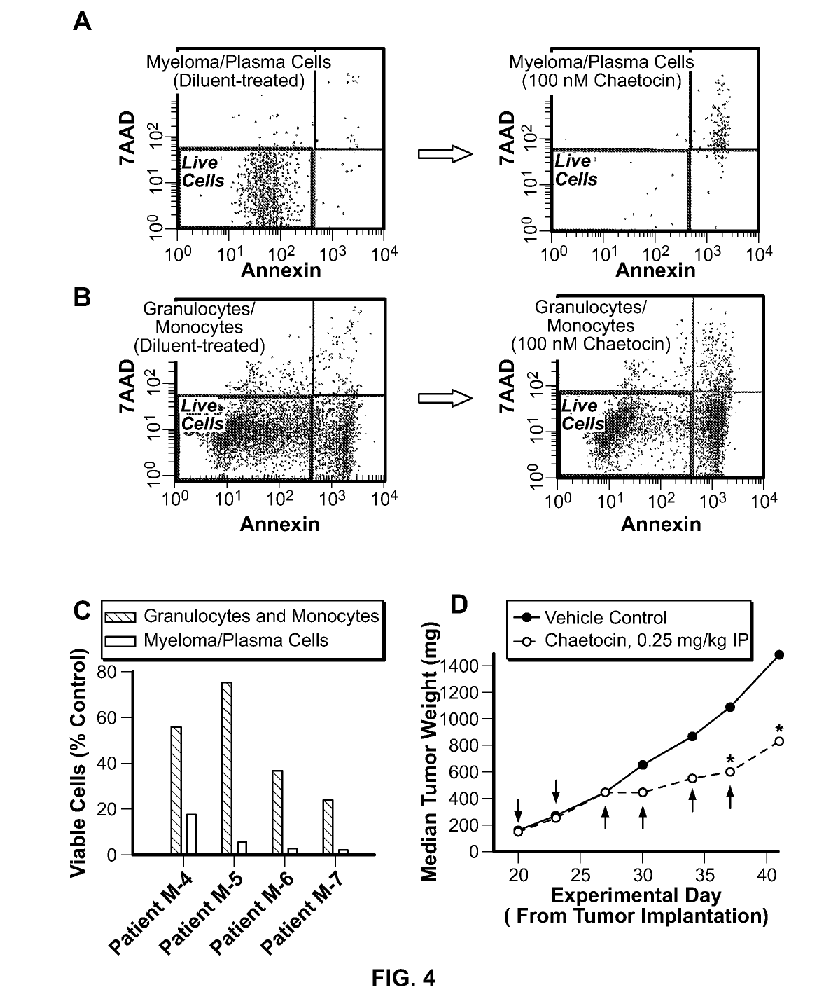

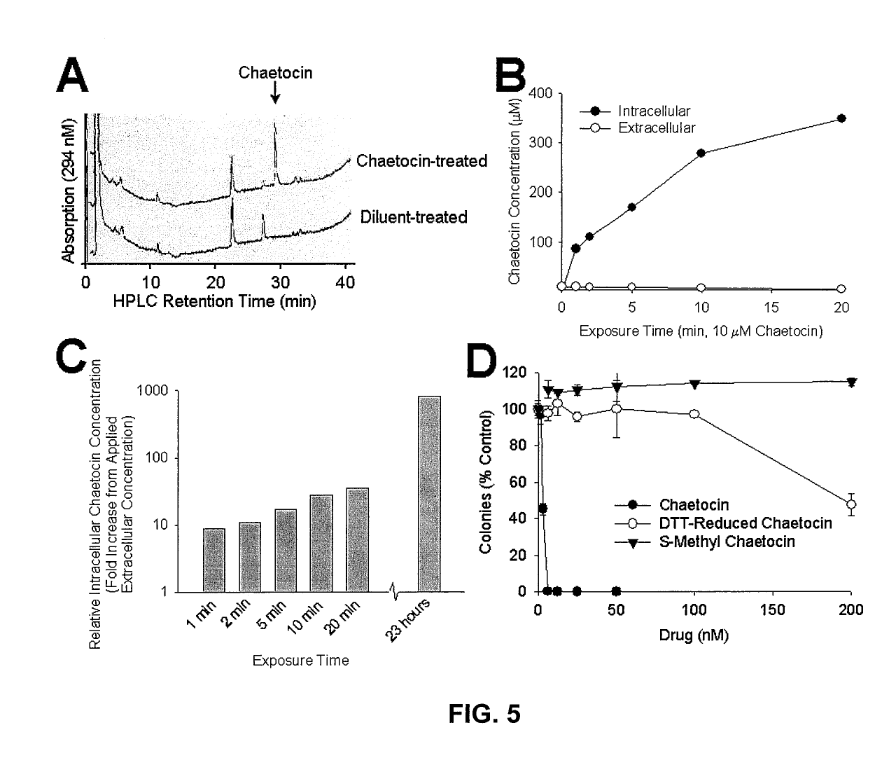

5. Whereas chaetocin potently and selectively kills sorted patient myeloma cells, it largely spares closely lineage-related normal B-lymphocytes and neoplastic B-CLL (chronic lymphocytic leukemia) cells. To further examine the selectivity of chaetocin-induced cytotoxicity in patient hematological cells more closely lineage-related to myeloma/plasma cells, the effects of chaetocin in sorted normal patient B-lymphocytes and neoplastic B-CLL cells (myeloma/plasma cells are of B-cell lineage) were examined. Impressively, chaetocin did not differentially kill sorted patient normal B-lymphocytes (FIG. 3D) or sorted patient neoplastic B-CLL cells (FIG. 3E), indicating a high degree of selectivity of the cytotoxic effects of chaetocin even in very closely lineage-related normal and neoplastic B-cells.
6. Chaetocin potently kills patient myeloma cells when treated in mixed bone marrow cultures. Over concern that the observed cytotoxic effects of chaetocin might reflect an artifact seen only in sorted myeloma cells, chaetocin-induced cytotoxicity in myeloma cells and neutrophils/monocytes treated in mixed bone marrow culture was evaluated. Unsorted bone marrow cells obtained from relapsed myeloma patients were treated with chaetocin (100 nM) or diluent in mixed culture (24 hours), with the cytotoxic effects of chaetocin evaluated using multi-channel FACS analyses. Consistent with the selectivity observed in sorted cells, chaetocin also killed myeloma cells with selectivity in comparison to control patient neutrophils/monocytes in this mixed culture system (FIG. 4A-C).
7. Chaetocin has in vivo anti-myeloma activity. Preliminary in vivo experiments using chaetocin (0.25 mg/kg intraperitoneally twice weekly, RPMI 8226 SCID mouse model; flank xenografts) demonstrated antiproliferative activity (p<0.05), with T/C values in the 50-60% range in response to three weeks of treatment of established tumors (FIG. 4D).
8. Chaetocin is rapidly and dramatically accumulated in cancer cells by means that require intact/unreduced chaetocin disulfide bonds. Given the promising in vitro, ex vivo and in vivo anti-myeloma activity of chaetocin, detailed studies of its cellular handling and mechanisms of action were undertaken. As the related compound gliotoxin is concentrated in mammalian cells, chaetocin was evaluated to see whether it might be subject to intracellular accumulation in myeloma cell lines, freshly collected patient myeloma cells, and A549 human non small cell carcinoma cells. Results were similar for all examined cell types. Real-time assessment of intracellular chaetocin levels using HPLC (FIG. 5A) demonstrated rapid and dramatic dose-dependent intracellular accumulation of chaetocin in unmodified form, with simultaneous decrement of chaetocin concentrations in media (FIG. 5B). Impressively, intracellular accumulation of chaetocin reached 10-fold higher than applied extracellular concentration within 1-2 minutes, and up to 800-1000-fold higher than applied levels at 24 hours (FIGS. 5B and C).

Because of reports indicating that intracellular accumulation of the structurally related thiodioxopiperazine gliotoxin might be attenuated by reduction of its disulfide (Bernardo P H, Brasch N, Chai C L, and Waring P, A novel redox mechanism for the glutathione-dependent reversible uptake of a fungal toxin in cells, J Biol. Chem. (2003) 278:46549-46555), the effects of reduction of chaetocin disulfides with dithiothreitol (DTT) on chaetocin-induced reductions in colony formation as well as on the intracellular accumulation of derivatized chaetocin was evaluated. Reduction of chaetocin disulfides dramatically attenuated chaetocin-induced cytotoxicity (FIG. 5D, open circles), prompting more definitive evaluations examining the effects of S-methylation of the chaetocin disulfides on cytotoxicity (S-methylation prohibits equilibrium/spontaneous reformation of the chaetocin disulfide bonds). Consistent with an important role of the intact chaetocin disulfides on intracellular accumulation, S-methylation of chaetocin resulted in complete abrogation of its cytotoxicity (FIG. 5D, filled triangles); with cellular uptake studies demonstrating that S-methyl chaetocin was not accumulated in cancer cells (data not shown)—hence, providing an explanation for its lessened cytotoxicity and indicating the requirement of intact chaetocin disulfide bonds for cellular uptake/accumulation.

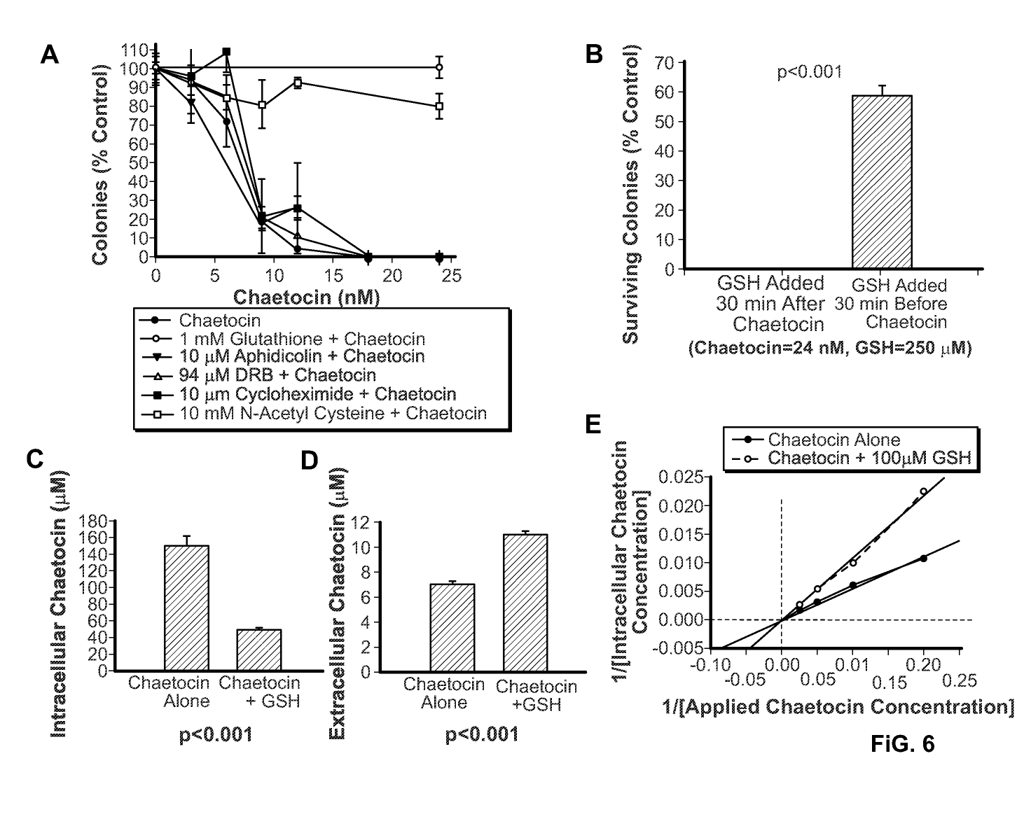

9. Glutathione pretreatment attenuates chaetocin-induced cytotoxicity and impairs cellular accumulation of chaetocin. The effects of glutathione (the primary intracellular reductant), N-acetyl cysteine (NAC, a cell-permeable glutathione precursor), aphidicolin (an inhibitor of DNA synthesis), DRB (an inhibitor of RNA synthesis), or cycloheximide (an inhibitor of protein synthesis) on the ability of chaetocin to inhibit colony formation in A549 cells were examined. Only NAC and glutathione attenuated chaetocin-induced cytotoxicity (FIG. 6; results recapitulated in myeloma cell lines, data not shown), while neither agent reduced chaetocin disulfide bonds in culture media (assessed using HPLC).

To determine whether agents that inhibit HDACs might similarly induce cytotoxicity via imposition of cellular oxidative stress, the effects of NAC on reductions in A549 colony formation resulting from treatment with the HDAC inhibitors apicidin, LAQ-824, tricostatin A or valproic acid was examined. In each case, colony formation was unaffected by NAC (data not shown), reinforcing the hypothesis that chaetocin-induced cytotoxicity is attributable to effects other than upon HDACs.

Notably, the cytoprotective effects of glutathione required its application before, or very shortly after, initiation of chaetocin exposure (FIG. 6B), prompting examination of whether glutathione might inhibit chaetocin cellular uptake (as it did not reduce chaetocin disulfides). Indeed, glutathione pretreatment attenuated the cellular accumulation of chaetocin (FIGS. 6C and D), with preliminary kinetic evaluation suggesting that glutathione and chaetocin may interact with the same cellular transport system (FIG. 6E). However, the extent to which glutathione blunted intracellular chaetocin accumulation (FIG. 6C) was modest in comparison to the magnitude of its effects on chaetocin-induced cytotoxicity (FIG. 6A), leading to the hypothesis that additional effects of glutathione might also be contributory to its cytoprotective effects. Because both glutathione and NAC are known to attenuate levels of cellular oxidative stress, whether these agents might exert cytoprotective effects via opposing cytotoxic cellular oxidative stress putatively imposed by chaetocin was examined.

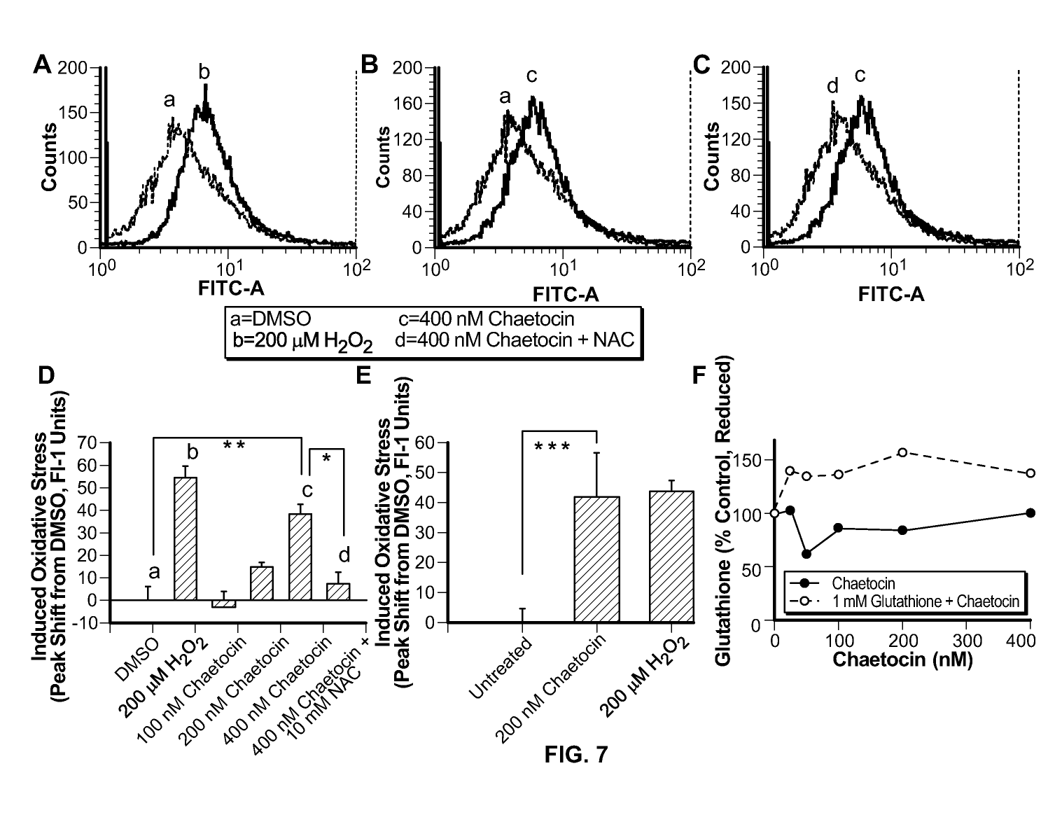

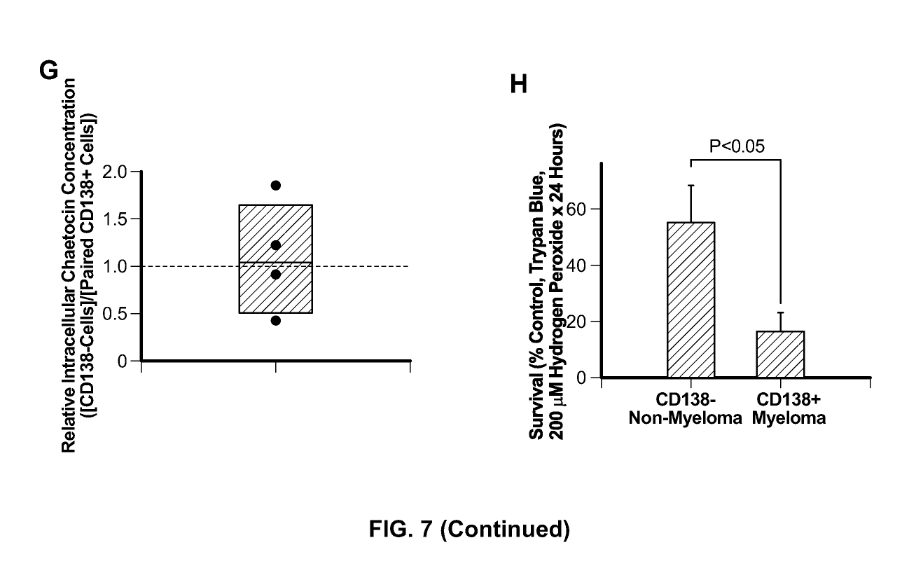

10. Chaetocin-induced cytotoxicity is associated with the induction of oxidative stress without depletion of intracellular levels of reduced glutathione. FACS analyses employing the oxidative stress-sensitive probe, 5,6-carboxy-2′,7′-difluoro-dihydro-fluorescein diacetate demonstrated that chaetocin induced increases in levels of cellular oxidative stress in a dose-dependent fashion in A549 cells (FIG. 7D) much more potently than seen in response to peroxide treatment (FIGS. 7B and A respectively; FIG. 7D). Further, NAC pretreatment attenuated chaetocin-induced oxidative stress (FIGS. 7C and D), concordant with its ability to attenuate chaetocin-induced cytotoxicity (FIG. 6A). Nevertheless, chaetocin did not appreciably deplete intracellular reduced glutathione (FIG. 7E), indicating that depletion of glutathione is apparently not involved in the imposition of cytotoxic cellular oxidative stress by chaetocin.

Unfortunately, available reagents for ROS quantitation including the oxidative stress-sensitive probe 5,6-carboxy-2′,7′-difluoro-dihydro-fluorescein diacetate are for unknown reasons not suitable in detection of ROS in myeloma cell lines, prompting the use of A549 cells for the above experiments. However, an alternative FACS probe, hydroethidium, is useful for assessment of ROS (superoxide in particular) in U266 myeloma cells. Experiments with this probe demonstrated that 200 nM chaetocin induced a peak shift of 43.26+3.67 (FIG. 7E, p<0.001) relative to stained, untreated U266 cells—confirming that ROS is also induced by chaetocin in myeloma cells.

11. Differential cytotoxicity observed between patient myeloma cells and paired normal patient leukocytes is not related to differential cellular accumulation of chaetocin. Having established that chaetocin is rapidly and dramatically accumulated in cancer cells—and that induction of oxidative stress in an important factor in chaetocin-induced cytotoxicity—how these factors might relate to the differential cytotoxicity observed between myeloma cells and normal bone marrow leukocytes was examined. First, evaluation of the levels of intracellular chaetocin resulting from treating freshly collected matched patient CD138+ and CD138− cells with 10 μM chaetocin for 20 minutes was undertaken using HPLC in samples from 4 myeloma patients. Differential chaetocin sensitivity of examined cells was evaluated via parallel assessment of the cytotoxic effects of treating each cell population with 100 nM chaetocin for 24 hours (data not shown, similar to FIG. 3A-C). After correction for average cell volumes, no indication of lessened intracellular accumulation of chaetocin in the less sensitive normal CD138− leukocytes was found (FIG. 7G), indicating that the observed heightened sensitivity of CD138+ myeloma cells to the cytotoxic effects of chaetocin is apparently not attributable to differential cellular accumulation of drug.
12. Differential chaetocin-induced cytotoxicity observed between patient myeloma cells and paired normal patient leukocytes may be attributable to a relative hypersensitivity of myeloma cells to imposed oxidative stress. Having found no indication that the heightened cytotoxicity of chaetocin in myeloma cells in comparison to matched normal leukocytes might be attributable to increased intracellular accumulation of chaetocin in the more sensitive myeloma cells, the inherent susceptibility of myeloma cells to the cytotoxic effects of oxidative stressors was examined. To preliminarily examine this possibility, the cytotoxic effects of another oxidative stressor, H₂O₂, as compared chaetocin in paired CD138+ myeloma cells and negatively sorted CD138− normal marrow leukocytes were examined. Indeed, not only were examined patient myeloma cells more sensitive to chaetocin than their normal marrow leukocyte counterparts (data not shown, results similar to FIG. 3A-C), but patient myeloma cells were also more sensitive to peroxide-induced cytotoxicity in comparison to matched normal patient leukocytes (FIG. 7H). Considered in aggregate, the observed selective cytotoxicity of chaetocin in myeloma cells therefore appears, at least in part, to be attributable to a generally heightened susceptibility of myeloma cells to oxidative stressors.
13. Chaetocin has potent and selective anti-cancer activity in solid tumor cell lines. Many solid tumor cell lines are readily killed by chaetocin (FIG. 10). Furthermore, chaetocin has been demonstrated to kill thyroid cancer cell lines more readily (selectively) than normal thyrocytes (normal thyroid cells, FIG. 12). This indicates that chaetocin has selectivity for cancerous cells in at least some solid tumor models.
14. Chaetocin is effective in killing even highly chemotherapy-resistant cancer cells and cell lines. Having seen that chaetocin readily kills freshly collected patient myeloma cells that are largely resistant to dexamethasone or doxorubicin (see FIG. 3A-C), the effects of chaetocin in dexamethasone- or doxorubicin-resistant myeloma cell lines were assessed. In particular, dexamethasone-resistant MM1R_{L} cells were found to be equally sensitive to chaetocin-induced cytotoxicity as were dexamethasone-sensitive MM1S cells, despite maintaining a high level of resistance to dexamethasone (FIG. 13), confirming no cross-resistance to chaetocin. PgP overexpressing 8226 D40 cells exhibited only modest cross-resistance to chaetocin in comparison to doxorubicin (FIG. 13). Hence, studies employing drug-resistant myeloma cell lines recapitulated ex vivo results to confirm that dexamethasone or doxorubicin resistant myeloma cells appear largely non-cross resistant to chaetocin.

### Results for Thioredoxin Reductase Experiments

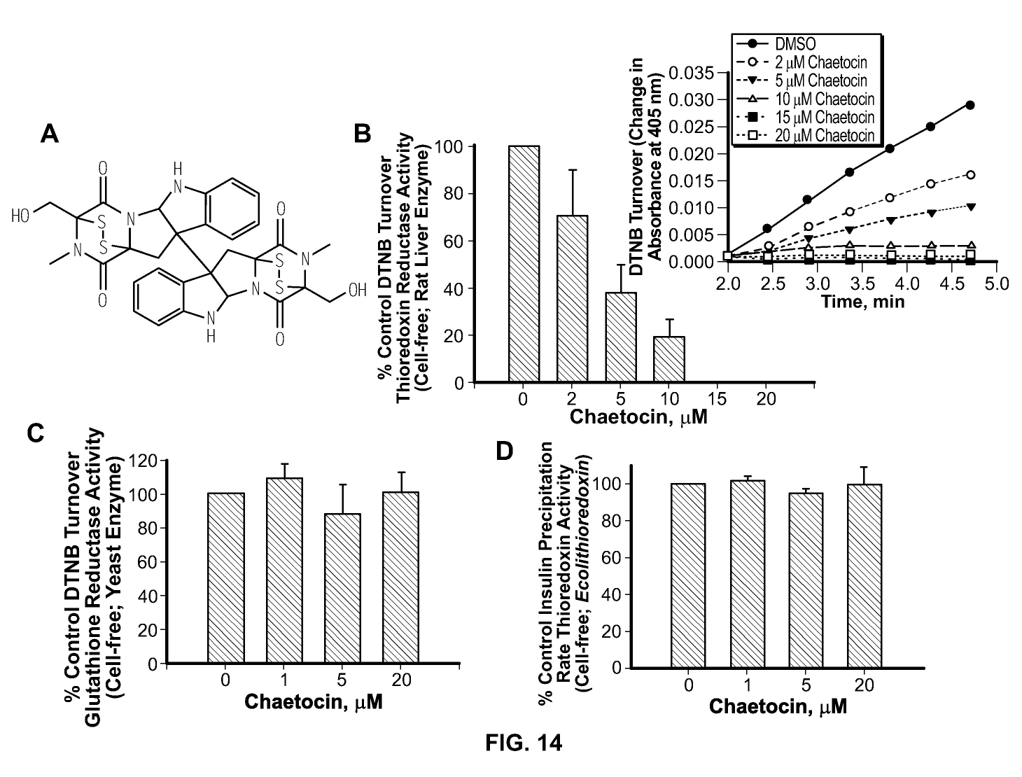

15. Chaetocin inhibits thioredoxin reductase but not glutathione reductase or thioredoxin. Since chaetocin (FIG. 8, FIG. 14A) contains two disulfide bonds and is known to induce oxidative stress in cancer cell, as shown above, chaetocin might interact with oxidative stress-related proteins that rely upon disulfide bond redox cycling for activity. Initial experiments showed that chaetocin inhibited TrxR1-initiated turnover of the synthetic substrate DTNB (Reaction 1) in a cell-free assay in a dose-responsive manner (FIG. 14), with an IC₅₀ of about 4 μM.

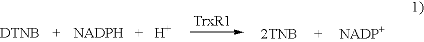

The activity of a related family member, glutathione reductase (G.R.; Reaction 2), however, was unaffected by up to 20 μM chaetocin (FIG. 14C).

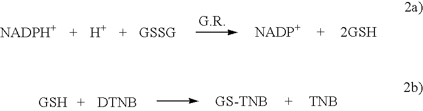

The effects of chaetocin on the reductase activity of Trx were also examined, since Trx is a major downstream effector substrate of TrxR1—and because Trx itself is a disulfide-containing reductase. Since chaetocin inhibits TrxR1 activity, however, an activity assay based on insulin reduction (Reaction 3) was utilized that did not rely on the coupled TrxR1/Trx reaction. Additionally, because DTT reduces disulfide bonds and has been previously shown to reduce chaetocin under certain conditions, the reduction state of chaetocin in the presence of DTT was also examined by mass spectrometry in response to Trx activity assay conditions to establish that under utilized reaction conditions, DTT itself did not appreciably reduce chaetocin (data not shown). Confident that chaetocin was not reduced by DTT and therefore not inactivated over the time-span of the utilized Trx activity assay, up to 20 μM chaetocin does not appreciably effect Trx activity (FIG. 14D). However, experiments with higher chaetocin concentrations demonstrated Trx activity inhibition with a IC₅₀ of about 90 μM, about 20 times the chaetocin concentration required to similarly inhibit TrxR1. Collectively, these results indicate that chaetocin inhibits TrxR1 activity with a high degree of selectivity when compared to even closely related reductases, and that chaetocin is therefore not an indiscriminate inhibitor of all disulfide-containing reductases.

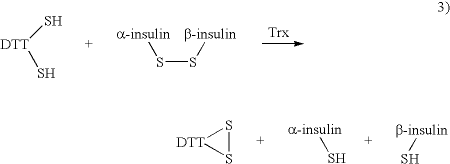

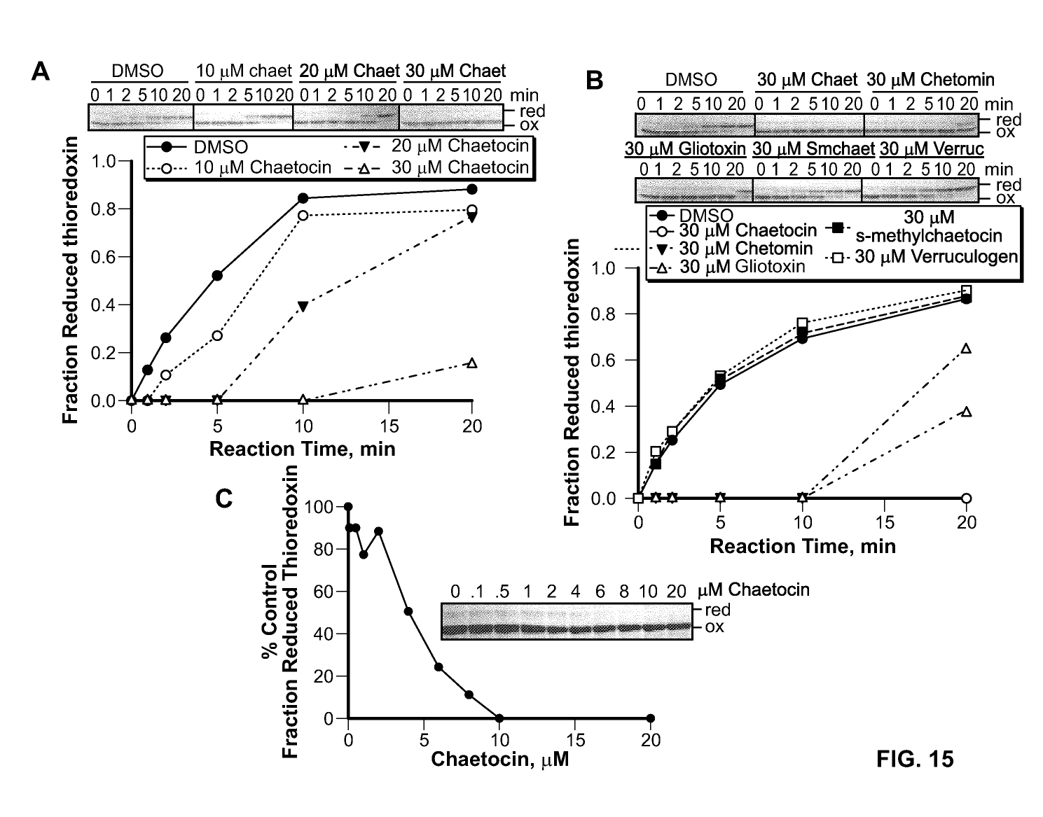

16. Chaetocin and related thiodioxopiperazines inhibit the reduction of thioredoxin by thioredoxin reductase. As the small molecule DTNB utilized in the previous assay is not the native substrate for thioredoxin reductase, chaetocin might also impair the ability of TrxR1 to reduce its native substrate, thioredoxin (Reaction 4). A novel gel-based kinetics assay was developed to resolve the oxidized and reduced forms of Trx by rapid covalent modification of the free sulfhydryl groups of Trx with AMS. Using this method, chaetocin was indeed also found to inhibit the ability of TrxR1 to reduce Trx (FIG. 15A) in a dose- and time-dependent fashion. In addition, the structurally related thiodioxopiperazines gliotoxin and chetomin also inhibited the activity of thioredoxin reductase, albeit somewhat less effectively than chaetocin (FIG. 15B). Not surprisingly, intact disulfide bonds of tested thiodioxopiperazines were found to be critical to this inhibition, based on the inability of S-methyl chaetocin or the reduced thiodioxopiperazine verruculogen to recapitulate this effect (FIG. 15B). Chaetocin's IC₅₀ for this reaction was found to be 4 μM under conditions optimized to be within the initial linear portion of the reaction (FIG. 15C).

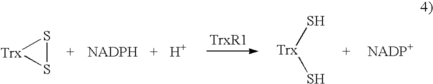

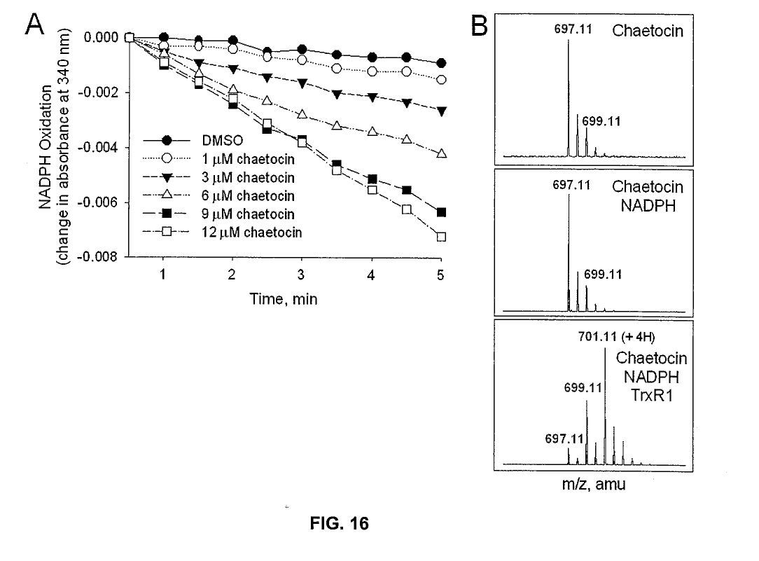

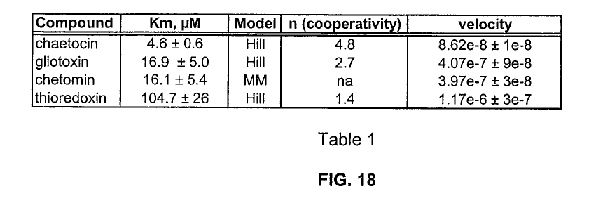

As achieved intracellular chaetocin concentrations are as high as 1000-fold greater than applied extracellular concentrations, cytotoxic intracellular chaetocin levels easily reach 25-100 times its IC₅₀ for this reaction (100-400 μM), underscoring the potential biological relevance of TrxR1 inhibition by chaetocin to its anticancer effects.
17. Chaetocin and related thiodioxopiperazines serve as substrates for thioredoxin reductase. Chaetocin, which itself contains two disulfides, might inhibit thioredoxin reductase by serving as a competitive substrate for TrxR1. Consistent with this possibility, NADPH is indeed oxidized over time when chaetocin is substituted for Trx in Reaction 4, with a Kₘ for chaetocin of 4.6±0.6 μM; indicative of substrate functionality (FIG. 16A). Interestingly, the Kₘ for Trx in this same assay is 104.7±26 μM, almost 25 times higher than that of chaetocin, indicating that chaetocin effectively serves as a more efficient substrate for TrxR1 than its native substrate, Trx. Presented data are therefore consistent with the hypothesis that the ability of chaetocin to serve as a TrxR1 inhibitor (FIG. 14) most likely relates to its function as a competitive TrxR1 substrate. The related compounds gliotoxin and chetomin have Kₘ's of 16.0±5.0 and 16.1±5.4 μM, respectively (FIG. 18, Table 1), indicating that related thiodioxopiperazines are also TrxR1 substrates, but that chaetocin is the highest affinity TrxR1 substrate of the series.

Confirmatory mass spectrometry analyses of chaetocin in the presence and absence of TrxR1 showed the expected parent peak of 697.11 m/z with chaetocin alone or after addition of NADPH. In the presence of thioredoxin reductase, however, a minor peak at 699.11 m/z (+2 Da; corresponding to the addition of two protons) and a major peak at 701.11 m/z (+4 Da; corresponding to the addition of four protons) were observed (FIG. 16B)—consistent with the hypothesis that the disulfide bonds of chaetocin are reduced by TrxR1, as expected if chaetocin serves as a substrate for the enzyme. Furthermore, no evidence of a covalent chaetocin-TrxR1 adduction was observed using mass spectroscopy (data not shown), in contrast to what has been reported in the instance of the interaction of sporidesmin with glutaredoxin (Srinivasan et al, Biochem., Vol. 45:8978-8987 (2006)).

18. Transient thioredoxin overexpression attenuates chaetocin-induced cell death.

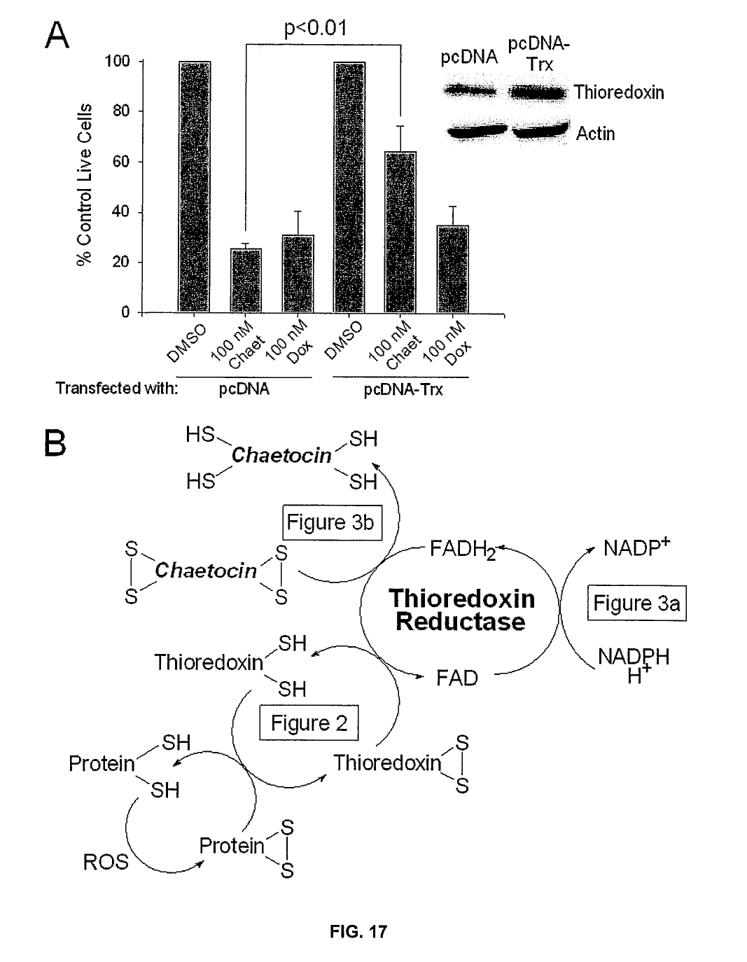

The above studies collectively indicate that, although serving as a TrxR1 competitive substrate, chaetocin does not in fact modify TrxR1 itself, but instead attenuates reduction of the downstream TrxR1 effector Trx. Consequently, it was hypothesized that Trx (and not TrxR1) ultimately functions as the primary cellular ROS-scavenger affected by chaetocin, Trx was transiently overexpressed as a means of exploring the potential linkage between the anticancer effects of chaetocin and its ability to inhibit TrxR1. Indeed, Trx overexpression significantly attenuated chaetocin-induced cell death (FIG. 17A), consistent with a linkage between the ability of chaetocin to inhibit the reduction of Trx by TrxR1 and chaetocin-induced cytotoxicity. As a negative control the impact of Trx overexpression on doxorubicin-induced cytotoxicity was evaluated, as doxorubicin-induced cytotoxicity was not attenuated by NAC co-treatment, indicating lesser contributions of ROS to doxorubicin-induced cytotoxicity consistent with induction of cytotoxicity instead via topoisomerase II inhibition. Importantly, parallel experiments demonstrated that Trx overexpression did not appreciably rescue cells from doxorubicin-induced cell death (FIG. 17A), indicating that the attenuation of chaetocin-induced cell death in response to transient Trx overexpression was apparently not simply due to the indiscriminate induction of pro-survival signaling, but instead to the effects of chaetocin on the TrxR1/Trx pathway.

### Discussion

Multiple myeloma is an incurable cancer characterized by the clonal proliferation of B-cell lineage plasma cells resulting in the production of monoclonal proteins in serum and/or urine, destructive boney lesions, and the deaths of about 12,000 individuals in the U.S. alone annually. Although increasing numbers of therapeutics are becoming available to treat this disease—with the potential for significant symptom palliation, induction of disease responses and prolongation of disease-free survival—available therapeutic approaches including peripheral blood stem cell transplantation and newer agents have, unfortunately, had only modest impact on patient overall survival in several randomized trials. As a consequence, there is still a need for improved anti-myeloma therapies.

As demonstrated above, chaetocin represents a promising agent for further development as an anti-myeloma therapeutic. Not only does chaetocin have potent in vitro anti-myeloma activity (FIGS. 1B and C, 2), but also striking ex vivo potency and selectivity (FIG. 3A-C and 4); as well as in vivo efficacy (FIG. 4D). Moreover, studies of chaetocin in dexamethasone- or doxorubicin-resistant myeloma cell lines indicated that several highly drug-resistance myeloma lines were largely non-cross-resistant to chaetocin (FIG. 13). The selective ex vivo anti-myeloma effects of chaetocin are impressive even when compared to standard first line anti-myeloma agents such as dexamethasone and doxorubicin (FIG. 3A-C). Chaetocin exhibits striking selectivity in killing myeloma cells even in comparison to closely lineage-related normal and neoplastic B-lymphocytes (FIGS. 3D and E). Additionally, the striking anti-myeloma activity of chaetocin was observed in samples obtained from patients afflicted with many types of myeloma, including smoldering myeloma as well as heavily pretreated myeloma patients who had previously undergone peripheral blood stem cell transplantation. Myeloma cells from patients who had previously received “newer” therapeutics such as thalidomide or bortezomib—and those obtained from patients with a diverse array of cytogenetic abnormalities—were readily and selectively killed by chaetocin.

Mechanistically, it is also noteworthy that chaetocin-induced cytotoxicity may rely upon both intracellular drug accumulation (FIG. 5) and upon induction of cytotoxic oxidative stress upon cell entry (FIGS. 7A-F). The ability of chaetocin to selectively kill myeloma cells appears may be based upon a generally increased susceptibility of myeloma cells to oxidative stressors (FIG. 7 G, H).

While not being bound by theory, the present studies of chaetocin also indicate that intact chaetocin disulfide bonds may be required for its cellular entry, and therefore for its cytotoxicity (FIG. 4D). Unlike gliotoxin, intracellular reduction of chaetocin disulfides does not appear to take place to any appreciable extent (FIG. 5A). It therefore appears that intracellular reduction of thiodioxopiperazines disulfide bonds may not be required for the maintenance of high intracellular thiodioxopiperazine levels. Further, intact thiodioxopiperazine disulfides appear not to be directly responsible for cytotoxicity, as the gliotoxin disulfide apparently exists in the reduced state intracellularly, yet gliotoxin is still cytotoxic. As a consequence, thiodioxopiperazine structural moieties other than intact disulfides may be important in the infliction of cytotoxicity once cellular entry is otherwise secured. Based upon the observation that chaetocin-induced cytotoxicity appears to be dependant upon the induction of oxidative stress (FIGS. 6A and 7), the quinone-like carbonyl residues of chaetocin may be important in the induction of cellular oxidative stress, much akin to that imposed by many true quinines. Alternatively, it is also possible that the disulfides of thiodioxopiperazines may represent reactive sulfur species that are involved in the imposition of observed oxidative stress.

Although results indicating that glutathione or NAC co-treatment abrogates chaetocin-induced cytotoxicity (FIG. 6A) might ordinarily be interpreted as clear evidence that oxidative stress is involved, the reality of the situation may be significantly more complicated. In addition to alleviating oxidative stress, glutathione co-treatment also led to attenuated intracellular accumulation of chaetocin—perhaps via interaction with the transport system responsible for chaetocin cellular accumulation (FIG. 6C-E). Although it is interesting to speculate that chaetocin may interact with a glutathione transporter, little is currently known about glutathione transport—and it is generally believed that glutathione is poorly cell permeable. It may be instead, therefore, that glutathione itself is not the primary subject of the involved transport system, but instead a regulator of the system that so happens to facilitate intracellular accumulation of chaetocin.

Notwithstanding the ability of glutathione to attenuate the intracellular uptake of chaetocin, it was also observed that glutathione-induced attenuation of intracellular chaetocin accumulation (FIG. 6C) alone was not sufficient to account for the observed attenuation of chaetocin-induced cytotoxicity (FIG. 6A). Indeed, chaetocin was also found to induce oxidative stress in tumor cells more potently than that attained by treatment with H₂O₂ (FIG. 7A-D). Hence, glutathione co-treatment appears to attenuate chaetocin-induced cytotoxicity via two means: 1) attenuation of intracellular accumulation of chaetocin (FIG. 6C-E) and 2) attenuation of chaetocin-induced oxidative stress (FIG. 7A-D), presumably in part consequent to its ability to produce increased levels of reduced intracellular glutathione (FIG. 7E).

While no evidence that chaetocin might be selectively accumulated in myeloma cells relative to less sensitive normal leukocytes was observed (FIG. 7G), myeloma cells were not only more sensitive to chaetocin, but also more sensitive to the oxidative stressor H₂O₂ (FIG. 7H). Hence, the observed selective cytotoxic effects of chaetocin in myeloma cells may be attributable to a generally heightened sensitivity of myeloma cells to oxidative stress. Interestingly, there are data indicating that cancer cells are, overall, more susceptible to the cytotoxic effects of oxidative stressors, leading to the hypothesis that agents that induce cellular oxidative stress may be generally effective as cancer therapeutics. In summary, the results herein demonstrate that the thiodioxopiperazine natural product chaetocin has potent and selective in vitro, ex vivo, and in vivo anti-myeloma activity that appears to require both intact chaetocin disulfides to facilitate intracellular accumulation as well as the infliction of cellular oxidative stress upon cell entry.

### Discussion Related to Thiodoxin Reductase Experiments

This document presents data linking the effects of chaetocin on a specific molecular target, TrxR1 (FIGS. 14-16 and Table 1), to its ability to impose cellular oxidative stress and induce death in cancer cells (FIG. 17). It is believed that this document represents the first report to identify a specific molecular target of chaetocin to be causally linked to its antineoplastic effects. Moreover, not only chaetocin, but also several other structurally related thiodioxopiperazines including gliotoxin and chetomin, competitively inhibit TrxR1—suggesting that thiodioxopiperazines as a class may target TrxR1. Finally, in addition to providing important insights into the cytotoxic molecular targets of chaetocin and related thiodioxopiperazines, this document also highlights the potential utility of the TrxR-1/Trx pathway as an important source of promising candidate antineoplastic molecular targets.

Although presented herein is compelling evidence that chaetocin is a competitive substrate and inhibitor of TrxR-1, while not as potently inhibiting several related reductases including glutathione reductase and thioredoxin, it is always possible that chaetocin might in addition inhibit other yet untested reductases. The fact that intracellular chaetocin concentrations reach >100 μM clearly lends plausibility to this possibility, and data shown in FIG. 14 indicate that even Trx activity can be partially inhibited at achieved intracellular chaetocin concentrations. Therefore, this issue of whether inhibition of reductases in addition to TrxR1 might contribute to the biological activity of chaetocin remains an open question, with work to be undertaken to determine more comprehensively the effects of chaetocin across a wider spectrum of cellular reductases. Second, the effects that chaetocin might exert on other antioxidant systems such as glutathione, catalase, and/or superoxide dismutases (SODs) were explored—finding that although chaetocin does not deplete levels of reduced intracellular glutathione, exogenously applied catalase or forced SOD2 overexpression somewhat attenuate chaetocin-induced cytotoxicity. Although no evidence that chaetocin directly affects catalase or SOD2 activities has been observed, the possibility exists that effects of chaetocin on these other ROS mitigation enzymes might also contribute to its redox effects and investigation is currently underway. Third, it is of importance to note that others have identified chaetocin as an inhibitor of histone methyltransferase (HMT) H3:K9 via a high throughput screening initiative, and it is of course therefore possible that the effects of chaetocin on this other target might also contribute to its anticancer activity. Data described herein indicate that the cytotoxic effects of chaetocin in both lung cancer and myeloma cell lines is completely abrogated by co-treatment with the cell permeable reduced glutathione precursor N-acetyl cysteine (NAC). These observations are clearly more consistent with the hypothesis that chaetocin-induced cytotoxicity might be conferred via ROS imposition, than with the hypothesis that its cytotoxicity might instead be conferred by HMT H3:K9 inhibition. Nevertheless, attained micromolar chaetocin concentrations might easily allow it to have multiple molecular targets all in concert contributing to its anticancer effects.

While at first glance chaetocin might otherwise appear to be a classical inhibitor of TrxR1 (FIGS. 14 and 15), careful scrutiny indicated instead that chaetocin does not act as pure inhibitor of TrxR1, but rather as a competitive TrxR1 substrate (FIG. 16). Because chaetocin exhibits a significantly lower TrxR1 Kₘ than that of the TrxR1 native substrate Trx (Kₘ for chaetocin 4.6±0.6 μM vs. 104.7±26 μM for Trx), chaetocin effectively spends more time associated with the enzyme, consequently serving as a noncovalent TrxR1 inhibitor. This is supported by the observation that TrxR1 regains its activity at high chaetocin concentrations at later time points (FIGS. 15A and B), when chaetocin is completely reduced by TrxR1 and therefore no longer capable of TrxR1 inhibition as demonstrated for related compounds lacking intact bridged disulfide bonds (FIG. 15B; S-methyl chaetocin, verruculogen). These data bolster the contention that it is chaetocin's substrate functionality, and not other classical inhibitor qualities such as covalent binding or protein structure rearrangement, that ultimately results in the inhibition of TrxR1 by chaetocin. Furthermore, mass spectrometric analyses provide no hint of covalent modification of TrxR1 by chaetocin.

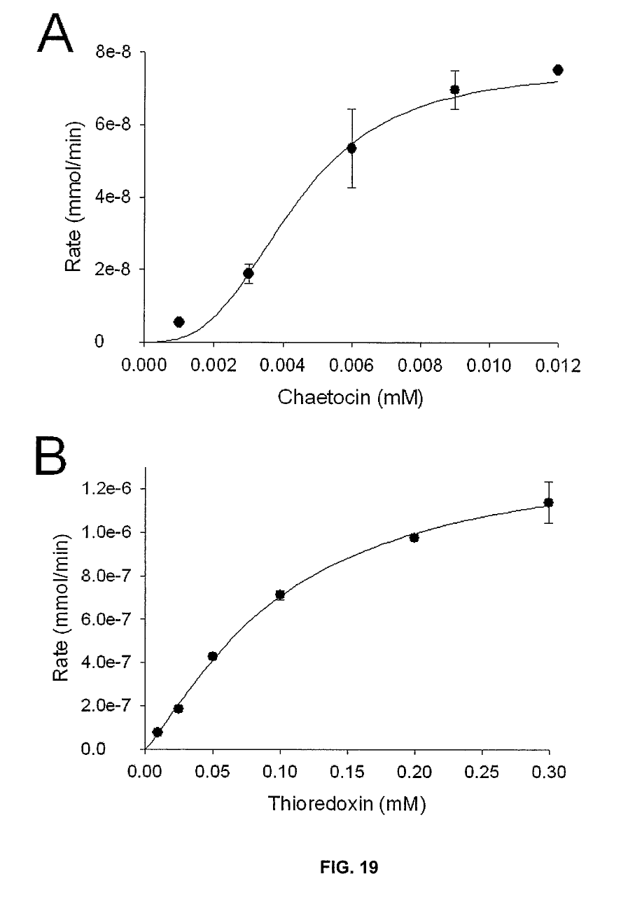

Human TrxR1 contains two redox sites, a Cys59-Cys64 active site pair, and a selenoCys 496′-Cys495′ pair in the C-terminal region that interacts with the active site cysteine pair. Glutathione reductase and Trx on the other hand each contain only solitary active sites, a cysteine-cysteine pair. Based on the ability of chaetocin to act as an substrate/inhibitor for TrxR1 but not G.R. or Trx, it is interesting to postulate that chaetocin might primarily interact with the C-terminal selenoCys 496′-Cys495′. Studies with TrxR1 mutants lacking the selenocysteine active site would be required to further examine this possibility. Also, since TrxR1 contains two active sites, it is intriguing that the initial rate (v) versus concentration [S] kinetics plots were best fit by the Hill equation for chaetocin and Trx (FIG. 19). This sigmoidal v by [S] plot often indicates cooperative binding of substrate to the active site. This behavior is most common for substrates interacting with multimeric enzymes containing several interacting active sites, and has been described for TrxR1 family members but not specifically for TrxR1 itself to the best of the inventors' knowledge.

Importantly, by inhibiting TrxR1, chaetocin consequently attenuates otherwise normal levels of TrxR1 redox cycling of its major downstream effector Trx (FIG. 15), thereby apparently compromising cellular ROS mitigation capacity, and thereby lending an explanation for the increased cellular ROS observed accompanying the exposure of cancer cells to chaetocin. Moreover, the observation that chaetocin-induced cytotoxicity is attenuated by Trx overexpression in cancer cells (FIG. 17A) importantly establishes a potential linkage between TrxR1 inhibition by chaetocin and chaetocin-induced ROS and anticancer activity. Because doxorubicin-induced cytotoxicity was unaffected by Trx overexpression (FIG. 17A), observed effects appear to reflect the specific targeting of TrxR1 by chaetocin, as opposed to nonspecific upregulation of survival signaling accompanying Trx overexpression.

It is noteworthy that inherently upregulated Trx overexpression in some cancers appears to convey worse prognosis, and also resistance to some conventional chemotherapeutics. This may be attributable to what has been referred to as oncogene addiction, whereby tumor cells overexpress a particular gene in order to gain a growth and/or survival advantage, but then go on to become dependent upon the consequently upregulated signaling. TrxR1 overexpression may in part represent an adaptive mechanism facilitating mitigation of otherwise cytotoxic higher basal ROS levels characteristic of many cancers, thereby making TrxR1 overexpression required for survival in these cancers. Consequently, agents like chaetocin that specifically target TrxR1 may represent especially attractive target-directed therapeutics for TrxR1—overexpressing neoplasms. Moreover, it is interesting to speculate that TrxR1 overexpression might be usable as a biomarker to define cancers most likely to respond to chaetocin therapy.

It is also noteworthy that in presented studies several thiodioxopiperazines (gliotoxin, chetomin) that are structurally related to chaetocin were also found to inhibit TrxR1, albeit somewhat less effectively than chaetocin (FIG. 15). This suggests that, as a class, thiodioxopiperazines, including disulfide bridged thiodioxopiperazines, may generally serve as TrxR1 competitive substrates and inhibitors. Of note, however, is that the structurally-related thiodioxopiperazine sporidesmin has previously been implicated as an inhibitor of glutaredoxin, yet was reported not to significantly inhibit TrxR1 (Srinivasan et al., Biochem., Vol. 45:8978-8987 (2006)). If indeed different thiodioxopiperazines have selectivity in inhibiting distinct cellular reductases, there may be further opportunities to develop members of this class of compounds as inhibitors of other reductases of potential relevance to cancer and other diseases.

In sum, thioredoxin reductase is a cytotoxic molecular target of chaetocin of potential relevance and importance to its selective anticancer effects. In particular, the data support a model (FIG. 17B) whereby chaetocin serves as a potent competitive substrate for the redox cycling enzyme thioredoxin reductase, competing with thioredoxin for reduction by TrxR1, and thereby serving to deplete levels of reduced cellular Trx, the critical ROS remediation substrate and downstream effector of TrxR1. Moreover, not only chaetocin, but also several other structurally related thiodioxopiperazines including gliotoxin and chetomin, competitively inhibit TrxR1—intimating that thiodioxopiperazines as a class may target TrxR1.

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising a compound according to Formula I: 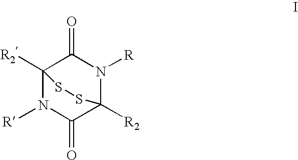
or a pharmaceutically acceptable salt or derivative thereof,
wherein R, R′, R₂, and R₂′, independently, can be selected from the group consisting of hydrogen, alkyl, cycloalkyl, alkoxy, hydroxy, aryl, heteroaryl, heteroalkyl, heterocyclyl, halo, pseudohalo, carboxy, haloalkyl, hydroxyalkyl, alkaryl, aralkyl, aminocarbonyl, aminoalkyl, alkylcarbonyl, arylcarbonyl, alkoxy, alkylthio, aryloxy, and arylthio; or
wherein R and R₂, and/or R′ and R₂′, independently, together to which the atoms to which they are attached, form a fused heterocyclic ring system which is optionally substituted with one or more alkyl, cycloalkyl, alkoxy, aryl, hydroxy, heteroaryl, heteroalkyl, heterocyclyl, halo, pseudohalo, carboxy, haloalkyl, hydroxyalkyl, alkaryl, aralkyl, aminocarbonyl, aminoalkyl, alkylcarbonyl, arylcarbonyl, alkoxy, alkylthio, aryloxy, or arylthio groups, or another optionally substituted aryl, heterocyclyl, or cycloalkyl fused ring system,
provided that R and R′ are not substituted with the same group when R₂ and R₂′ are both H, and provided that if R and R₂ form a fused ring heterocyclic ring system, then R′ and R₂′ do not also form a fused ring heterocyclic system, and if R and R₂ form a fused ring heterocyclic ring system and R′ is methyl, then R₂′ is not hydroxyalkyl or alkyl.

2. The composition according to claim 1, wherein R and R′ are selected independently from hydrogen, halo, pseudohalo, hydroxyl, alkyl having from 1 to 8 carbon atoms, aralkyl, where the alkyl group has from 1 to 8 carbon atoms, alkaryl, where the alkyl group has from 1 to 8 carbon atoms, hydroxyalkyl, where the alkyl group has from 1 to 8 carbon atoms, alkylcarbonyl, where the alkyl group has from 1 to 8 carbon atoms, and arylcarbonyl.

3. The composition according to claim 1, wherein R and R′ are independently selected from H, methyl, ethyl, hydroxymethyl, hydroxyethyl, and hydroxypropyl.

4. The composition according to claim 1, wherein R₂ and R₂′ are independently selected from hydrogen, halo, pseudohalo, alkyl having from 1 to 8 carbon atoms, and hydroxyalkyl, where the alkyl group has from 1 to 8 carbon atoms.

5. The composition according to claim 1, wherein R and R′ are H.

6. A composition of matter comprising a compound having the structure of chaetocin, chaetomin, or gliotoxin, or a pharmaceutically acceptable salt or derivative thereof, and an anticancer drug.

7. A method of treating or ameliorating one or more symptoms associated with cancer in a mammal, comprising administering a composition according to claim 1 or claim 6, or a composition comprising chaetocin, chaetomin, or gliotoxin, or a pharmaceutically acceptable salt or derivative thereof, to said mammal.

8. The method of claim 7, wherein said cancer overexpresses TrxR1.

9. The method of claim 7, wherein said cancer is a hematologic cancer or disorder.

10. The method of claim 9, wherein said cancer is multiple myeloma.

11. The method of claim 9, wherein said cancer is a B-lymphocyte lineage malignancy or disorder.

12. The method of claim 11, wherein said cancer is a leukemia or a lymphoma.

13. A method for inducing oxidative stress in a cancerous cell comprising contacting said cancerous cell with a composition according to claim 1 or claim 6, or a composition comprising chaetocin, chaetomin, or gliotoxin, or a pharmaceutically acceptable salt or derivative thereof.

14. The method of claim 13, wherein said cancer overexpresses TrxR1.

15. The method of claim 13, wherein said cancerous cell is a multiple myeloma cell.

16. A method for inducing apoptosis in a cancerous cell comprising contacting said cancerous cell with a composition according to claim 1 or claim 6, or a composition comprising chaetocin, chaetomin, or gliotoxin, or a pharmaceutically acceptable salt or derivative thereof.

17. A kit comprising a composition according to claim 1 or claim 6.

18. The kit of claim 17 wherein said composition is in the form of an injectible composition.

19. An article of manufacture comprising a composition according to claim 1 or claim 6, wherein said composition is provided for administration to a mammal in the form of a pill, a tablet, a capsule, or a syringe.

20. The method of claim 7, wherein said cancer is a solid tumorous cancer.

21. The method of claim 20, wherein said solid tumorous cancer is selected from the group consisting of lung, breast, prostate, hepatoma, thyroid, colon, cervical, pancreatic, and sarcoma solid tumors.

22. The method of claim 10, wherein said B-lymphocyte lineage malignancy or disorder is selected from MGUS, amyloidosis, heavy chain diseases, cryoglobulinemia, and Waldenstrom's macroglobulinemia.
